(19) United States
(12) Patent Application Publication
(10) Pub. No.: US 2022/0304993 A1
(43) Pub. Date: Sep. 29, 2022

(54) CONJUGATES OF AN ELECTRON-DONATING NITROGEN OR TERTIARY AMINE COMPRISING COMPOUNDS

(71) Applicant: Ascendis Pharma A/S, Hellerup (DK)

(72) Inventors: Nicola BISEK, Heidelberg (DE); Samuel WEISBROD, Heidelberg (DE)

(21) Appl. No.: 17/596,832

(22) PCT Filed: Jun. 19, 2020
(86) PCT No.: PCT/EP2020/067148

§ 371 Date: Dec. 20, 2021

(30) Foreign Application Priority Data
Jun. 21, 2019 (EP) 19181791.5

(51) Int. Cl. A61K 31/444; A61K 47/69; A61K 47/60; A61K 47/61; A61K 47/54

(52) U.S. Cl. CPC A61K 31/444; A61K 47/6903; A61K 47/60; A61K 47/61; A61K 47/545

(57) ABSTRACT

The present invention relates to conjugates of an electron-donating heteroaromatic nitrogen or tertiary amine comprising drugs and pharmaceutically acceptable salts thereof, pharmaceutical compositions comprising said conjugates and the use of said conjugates as medicaments.

The present invention relates to conjugates of an electron-donating heteroaromatic nitrogen or tertiary amine comprising drugs and pharmaceutically acceptable salts thereof, pharmaceutical compositions comprising said conjugates and the use of said conjugates as medicaments.

To improve physicochemical or pharmacokinetic properties, such as the in vivo circulation half-life of drugs, such drugs can be conjugated to a carrier, such as a polymer. Typically, polymers in drug delivery are either used in a non-covalent complexation of the drug and polymer, embedding of drug in a polymer or by covalent conjugation of the drug to a polymeric moiety.

However, the non-covalent approach requires a highly efficient drug encapsulation to prevent uncontrolled, burst-type release of the drug due to the disintegration of the drug-polymer complex after administration. Restraining the diffusion of an unbound, water-soluble drug molecule requires strong van der Waals contacts, frequently mediated through hydrophobic moieties and charged moieties for electrostatic binding. Many conformationally sensitive drugs, such as proteins or peptides, are rendered dysfunctional during the complexation process and/or during subsequent storage of the non-covalently bound drug.

Alternatively, a drug may be covalently conjugated to a polymeric moiety via a stable linker or a reversible linker from which the drug is released. If the drug is stably conjugated to the polymeric moiety, such conjugate needs to exhibit sufficient residual activity to have a pharmaceutical effect and thus the conjugate is constantly in an active form.

One advantage of conjugating a drug to a polymeric moiety through a reversible linker is that no residual activity of the conjugate is needed, because the drug exhibits its pharmacological effect upon release from the conjugate. A conjugate may exhibit no or little drug activity, i.e. the conjugate is pharmacologically inactive. This approach is applied to all classes of molecules, from so-called small molecules, through natural products up to large proteins. The drug of such a conjugate may be released by enzymatic or non-enzymatic cleavage of the linkage between the polymeric moiety and the drug moiety or by a combination of both.

Various reversible drug linkers are known in the art, for example WO 2005/099768 A2 discloses polymeric prodrugs, whereby amine comprising biologically active moieties are reversibly attached to transient linkers through, for example formation of amide or carbamate bonds. WO 2016/090050 A2 teaches antibody-drug-conjugates wherein attachment of the drug moiety to the linker results in formation of quaternary ammonium ions. WO 2017/205392 discloses pegylated carfilzomib conjugates, whereby carfilzomib is attached to the linker via formation of a quaternary ammonium ion.

Given the multitude of different drugs, it is desirable to have a large portfolio of reversible prodrug linkers available in order to identify the one that is most suitable for every type of conjugation chemistry and release half-life.

It is thus an object of the present invention to at least partially overcome the shortcomings described above.

This object is achieved with a conjugate or a pharmaceutically acceptable salt thereof comprising at least one moiety -D⁺ conjugated via at least one moiety -L¹-L²- to at least one moiety Z, wherein a moiety -L¹- is conjugated to a N⁺ of a moiety -D⁺ and wherein the linkage between -D⁺ and -L¹- is reversible and wherein a moiety -L²- is conjugated to Z, wherein
each -D⁺ is independently an electron-donating heteroaromatic N⁺-comprising moiety or a quaternary ammonium cation comprising moiety of a drug D, wherein each D comprises an electron-donating heteroaromatic N or a tertiary amine;
each -L²- is independently a single bond or a spacer moiety;
each Z is independently a polymeric moiety or a C₈₋₂₄ alkyl;
each -L¹- is independently a linker moiety of formula (I):

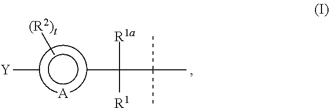

wherein
the dashed line indicates the attachment to the N⁺ of -D⁺;
t is selected from the group consisting of 0, 1, 2, 3, 4, 5 and 6;
-A- is a ring selected from the group consisting of monocyclic or bicyclic aryl and heteroaryl, provided that -A- is connected to —Y and —C(R¹)(R^{1a})— via carbon atoms; wherein said monocyclic or bicyclic aryl and heteroaryl are optionally substituted with one or more —R², which are the same or different; —R¹, —R^{1a} and each —R² are independently selected from the group consisting of —H, —C(O)OH, -halogen, —NO₂, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})— and —OC(O)N(R⁴)—;
   each -T- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -T- is independently optionally substituted with one or more —R³, which are the same or different;
   wherein —R³ is selected from the group consisting of —H, —NO₂, —OCH₃, —CN, —N(R⁴)(R^{4a}), —OH, —C(O)OH and C₁₋₆ alkyl; wherein C₁₋₆ alkyl is optionally substituted with one or more halogen, which are the same or different;
wherein —R⁴ and —R^{4a} are independently selected from the group consisting of —H and C₁₋₆ alkyl; wherein C₁₋₆ alkyl is optionally substituted with one or more halogen, which are the same or different;
—Y is selected from the group consisting of:

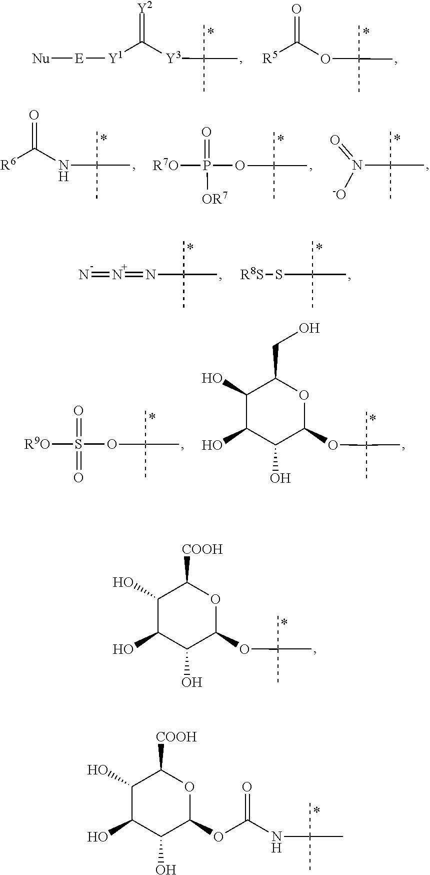

and a peptidyl moiety;
   wherein
   the dashed line marked with an asterisk indicates the attachment to -A-;
   -Nu is a nucleophile;
   —Y¹— is selected from the group consisting of —O—, —C(R¹⁰)(R^{10a})—, —N(R¹¹)— and —S—;
   ═Y² is selected from the group consisting of ═O, ═S and ═N(R¹²);
   —Y³— is selected from the group consisting of —O—, —S— and —N(R¹³)—;
   -E- is selected from the group consisting of C₁₋₆ alkyl, C₂₋₆ alkenyl, C₂₋₆ alkynyl and -Q-; wherein C₁₋₆ alkyl, C₂₋₆ alkenyl, C₂₋₆ alkynyl are optionally substituted with one or more —R¹⁴, which are the same or different;
   —R⁵, —R⁶, each —R⁷, —R⁸, —R⁹, —R¹⁰, —R^{10a}, —R¹¹, —R¹² and —R¹³ are independently selected from the group consisting of C₁₋₂₀ alkyl, C₂₋₂₀ alkenyl, C₂₋₂₀ alkynyl and -Q; wherein C₁₋₂₀ alkyl, C₂₋₂₀ alkenyl and C₂₋₂₀ alkynyl are optionally substituted with one or more —R¹⁴, which are the same or different; and wherein C₁₋₂₀ alkyl, C₂₋₂₀ alkenyl and C₂₋₂₀ alkynyl are optionally interrupted by one or more groups selected from the group consisting of -Q-, —C(O)O—, —O—, —C(O)—, —C(O)N(R¹⁵)—, —S(O)₂N(R¹⁵)—, —S(O)N(R¹⁵)—, —S(O)₂—, —S(O)—, —N(R¹⁵)S(O)₂N(R^{15a})—, —S—, —N(R¹⁵)—, —OC(OR¹⁵)R^{15a}—, —N(R¹⁵)C(O)N(R^{15a})— and —OC(O)N(R¹⁵)—;
   each Q is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q is independently optionally substituted with one or more —R¹⁴, which are the same or different;
   wherein —R¹⁴, —R¹⁵ and —R^{15a} are independently selected from the group consisting of —H and C₁₋₆ alkyl; wherein C₁₋₆ alkyl is optionally substituted with one or more halogen, which are the same or different; and
each -L¹- is substituted with -L²- and optionally further substituted.

It was surprisingly found that the reversible drug linker moiety -L¹- of formula (I) has advantageous properties, such as providing suitable release half-lives for drug moieties that are attached to said reversible linker moieties via quaternary ammonium cations. This is surprising as such electron-donating heteroaromatic N⁺-comprising moieties or quaternary ammonium cation comprising moieties may be good leaving groups that can be expected to result in half-lives that are unsuitable for reducing the frequency of drug administration.

Within the meaning of the present invention the terms are used as follows.

As used herein, the term “an electron-donating heteroaromatic N⁺-comprising moiety or a quaternary ammonium cation comprising moiety” refers to the moiety which after cleavage of the linkage between -D⁺ and -L¹- results in a drug D and wherein the drug moiety -D⁺ comprises at least one, such as one, two, three, four, five, six, seven, eight, nine or ten electron-donating heteroaromatic N⁺ or quaternary ammonium cations and analogously the corresponding D comprises at least one, such as one, two, three, four, five, six, seven, eight, nine or ten electron-donating heteroaromatic N or tertiary amines. Examples of chemical structures including heteroaromatic nitrogens i.e. N⁺ or N, that donate an electron to the aromatic π-system include, but are not limited to, pyridine, pyridazine, pyrimidine, quinoline, quinazoline, quinoxaline, pyrazole, imidazole, isoindazole, indazole, purine, tetrazole, triazole and triazine. For example, in the imidazole ring below the heteroaromatic nitrogen which donates one electron to the aromatic π-system is marked with “§”:

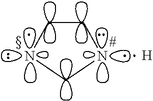

Such electron-donating heteroaromatic nitrogen atoms do not comprise heteroaromatic nitrogen atoms which donate one electron pair (i.e. not one electron) to the aromatic π-system, such as for example the nitrogen that is marked with “#” in the abovementioned imidazole ring structure. The drug D may exist in one or more tautomeric forms, such as with one hydrogen atom moving between at least two heteroaromatic nitrogen atoms. In all such cases, the linker moiety is covalently and reversibly attached at a heteroaromatic nitrogen that donates an electron to the aromatic π-system.

As used herein, the term “drug” refers to a substance used in the treatment, cure, prevention or diagnosis of a disease or used to otherwise enhance physical or mental well-being of a patient.

If a drug is conjugated to another moiety, the moiety of the resulting product that originated from the drug is referred to as “drug moiety”.

As used herein, the term “moiety” means a part of a molecule, which lacks one or more atom(s) or one or more electrons compared to the corresponding reagent. If, for example, a reagent of the formula “H—X—H” or “═X—” reacts with another reagent and becomes part of the reaction product, the corresponding moiety of the reaction product has the structure “H—X—”, “—X—” or “═X⁺—” whereas each “-” indicates attachment to another moiety. Accordingly, a drug moiety is released from a reversible linkage as a drug.

It is understood that if the chemical structure of a group of atoms is provided which group of atoms is attached to two moieties or is interrupting a moiety, said sequence or chemical structure can be attached to the two moieties in either orientation, unless explicitly stated otherwise. For example, a moiety “—C(O)N(R^{x})—” can be attached to two moieties or interrupting a moiety either as “—C(O)N(R^{x})—” or as “—N(R^{x})C(O)—”. Similarly, a moiety:

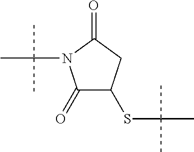

can be attached to two moieties or can interrupt a moiety either as

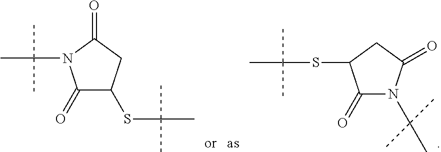

As used herein, the term “reagent” means a chemical compound, which comprises at least one functional group for reaction with the functional group of another chemical compound or drug. It is understood that a drug comprising a functional group is also a reagent.

It is recognized by one of ordinary skill in the art that the conjugates of the present invention are prodrugs. As used herein, the term “prodrug” refers to a drug moiety, that is reversibly and covalently conjugated to a polymeric moiety, such as Z, through at least one -L¹-L²- moiety. A prodrug releases the reversibly and covalently bound drug moiety -D⁺ in the form of its corresponding drug D. In other words, a prodrug is a conjugate comprising a drug moiety, which is covalently and reversibly conjugated to a polymeric moiety via at least one -L¹-L²-, moiety. Such prodrugs or conjugates release the formerly conjugated drug moiety in the form of a free drug.

As used herein, the term “reversible linkage” or “biodegradable linkage” is a linkage that is cleavable, in the absence or presence of enzymes under physiological conditions, which are aqueous buffer at pH 7.4 and 37° C., with a half-life ranging from one hour to six months, such as from one hour to four months, such as from one hour to three months, from one hour to two months or from one hour to one month. It is understood, however, that a reversible linkage may also be cleavable at other conditions, such as for example at a different pH or at a different temperature with a half-life ranging from one hour to six months, but that a test for determining reversibility is performed in the above-described physiological conditions (aqueous buffer, pH 7.4, 37° C.). Accordingly, a “stable linkage” is a linkage having a half-life under physiological conditions of more than six months.

As used herein, the term “reversible linker moiety” is a moiety which is covalently conjugated to a drug moiety through a reversible linkage and which is also covalently conjugated to a moiety Z via a moiety -L²-. In certain embodiments, the linkage between Z and -L²- is a stable linkage.

As used herein, the term “about” in combination with a numerical value is used to indicate a range ranging from and including the numerical value plus and minus no more than 10% of said numerical value, in certain embodiments, no more than 8% of said numerical value, in certain embodiments, no more than 5% of said numerical value and in certain embodiments, no more than 2% of said numerical value. For example, the phrase “about 200” is used to mean a range ranging from and including 200+/−10%, i.e. ranging from and including 180 to 220; in certain embodiments, 200+/−8%, i.e. ranging from and including 184 to 216; in certain embodiments, ranging from and including 200+/−5%, i.e. ranging from and including 190 to 210; and in certain embodiments 200+/−2%, i.e. ranging from and including 196 to 204. It is understood that a percentage given as “about 20%” does not mean “20%+/−10%”, i.e. ranging from and including 10 to 30%, but “about 20%” means ranging from and including 18 to 22%, i.e. plus and minus 10% of the numerical value which is 20.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined below. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—, —CH(C₂H₅)— and —C(CH₃)₂—. Each hydrogen atom of a C₁₋₆ carbon may optionally be replaced by a substituent as defined below. Optionally, a C₁₋₆ alkyl may be interrupted by one or more moieties as defined below.

Accordingly, “C₁₋₁₀ alkyl”, “C₁₋₂₀ alkyl”, “C₈₋₂₄ alkyl” or “C₁₋₅₀ alkyl” means an alkyl chain having 1 to 10, 1 to 20, 8 to 24 or 1 to 50 carbon atoms, respectively, wherein each hydrogen atom of the C₁₋₁₀, C₁₋₂₀, C₈₋₂₄ or C₁₋₅₀ carbon may optionally be replaced by a substituent as defined below. Optionally, a C₁₋₁₀ alkyl, C₁₋₂₀ alkyl, C₈₋₂₄ alkyl or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example of such C₂₋₆ alkenyl is —CH═CH—. Each hydrogen atom of a C₂₋₆ alkenyl moiety may optionally be replaced by a substituent as defined below. Optionally, a C₂₋₆ alkenyl may be interrupted by one or more moieties as defined below.

Accordingly, the terms “C₂₋₁₀ alkenyl”, “C₂₋₂₀ alkenyl” or “C₂₋₅₀ alkenyl” alone or in combination mean a straight-chain or branched hydrocarbon moiety comprising at least one carbon-carbon double bond having 2 to 10, 2 to 20 or 2 to 50 carbon atoms, respectively. Each hydrogen atom of a C₂₋₁₀ alkenyl, C₂₋₂₀ alkenyl or C₂₋₅₀ alkenyl group may optionally be replaced by a substituent as defined below. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C—CH₃. When two moieties of a molecule are linked by the alkynyl group, then an example is —C≡C—. Each hydrogen atom of a C₂₋₆ alkynyl group may optionally be replaced by a substituent as defined below. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined below. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₈₋₂₄ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which in certain embodiments are selected from the group consisting of

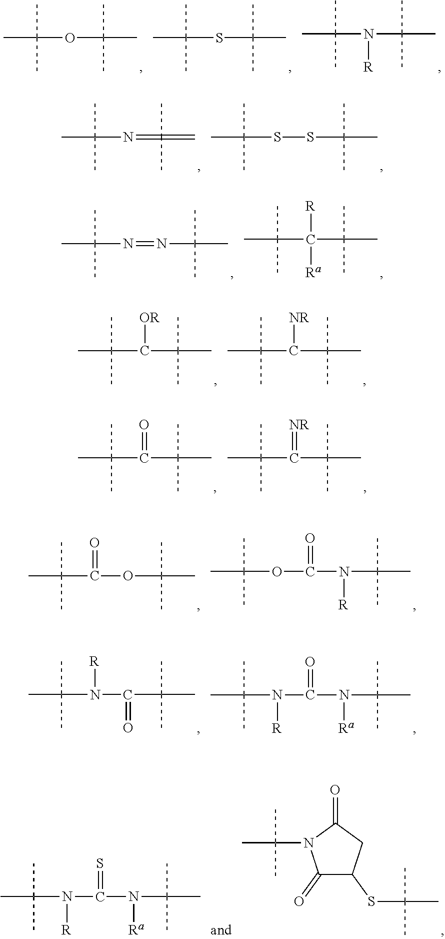

wherein
dashed lines indicate attachment to the remainder of the moiety or reagent; —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined below. The term “C₃₋₁₀ cycloalkyl” also includes bridged bicycles like norbornane or norbornene.

As used herein, the term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). In certain embodiments, an 8- to 30-membered carbopolycyclyl means a cyclic moiety of two, three, four or five rings. In certain embodiments, an 8- to 30-membered carbopolycyclyl means a cyclic moiety of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent as defined below.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent as defined below.

Similarly, the term “8- to 30-membered heteropolycyclyl” or “8- to 30-membered heteropolycycle” means a heterocyclic moiety of more than two rings with 8 to 30 ring atoms, in certain embodiments of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

As used herein, the term “monocyclic or bicyclic aryl” means an aromatic hydrocarbon ring system which may be monocyclic or bicyclic, wherein the monocyclic aryl ring consists of at least 5 ring carbon atoms and may comprise up to 10 ring carbon atoms and wherein the bicyclic aryl ring consists of at least 8 ring carbon atoms and may comprise up to 12 ring carbon atoms. Each hydrogen atom of a monocyclic or bicyclic aryl may be replaced by a substituent as defined below.

As used herein, the term “monocyclic or bicyclic heteroaryl” means a monocyclic aromatic ring system that may comprise 2 to 6 ring carbon atoms and 1 to 3 ring heteroatoms or a bicyclic aromatic ring system that may comprise 3 to 9 ring carbon atoms and 1 to 5 ring heteroatoms, such as nitrogen, oxygen and sulfur. Examples for monocyclic or bicyclic heteroaryl groups include, but are not limited to, benzofuranyl, benzothiophenyl, furanyl, imidazolyl, indolyl, azaindolyl, azabenzimidazolyl, benzoxazolyl, benzthiazolyl, benzthiadiazolyl, benzotriazolyl, tetrazinyl, tetrazolyl, isothiazolyl, oxazolyl, isoxazolyl, pyrazinyl, pyrazolyl, pyridazinyl, pyridinyl, pyrimidinyl, pyrrolyl, quinolinyl, quinazolinyl, quinoxalinyl, triazolyl, thiazolyl and thiophenyl. Each hydrogen atom of a monocyclic or bicyclic heteroaryl may be replaced by a substituent as defined below.

As used herein, the term “nucleophile” refers to a reagent or functional group that forms a bond to its reaction partner, i.e. the electrophile by donating both bonding electrons.

It is understood that the phrase “the pair —R^{x}/—R^{y} is joined together with the atom to which they are attached to form a C₃₋₁₀ cycloalkyl, 3- to 10-membered heterocyclyl or an 8- to 11-membered heterobicyclyl” in relation with a moiety of the structure:

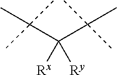

means that R^{x} and R^{y} form the following structure:

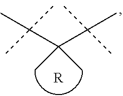

wherein R is C₃₋₁₀ cycloalkyl, 3- to 10-membered heterocyclyl or an 8- to 11-membered heterobicyclyl.

As used herein, the term “excipient” refers to a diluent, adjuvant or vehicle with which the therapeutic, such as a drug or conjugate, is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, hyaluronic acid, propylene glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid) or can contain detergents, like Tween®, poloxamers, poloxamines, CHAPS, Igepal® or amino acids like, for example, glycine, lysine or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or drug moiety, together with a suitable amount of excipient so as to provide the form for proper administration to the patient. The formulation should suit the mode of administration.

As used herein, the term “free form” of a drug refers to the drug in its unmodified, pharmacologically fully active form, e.g. after being released from the conjugate.

As used herein, the term “functional group” means a group of atoms which can react with other groups of atoms. Exemplary functional groups are carboxylic acid, primary amine, secondary amine, tertiary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane and aziridine.

As used herein, the term “halogen” means fluoro, chloro, bromo or iodo. In certain embodiments, halogen is fluoro or chloro.

As used herein, the term “interrupted” means that a moiety is inserted in between two carbon atoms or—if the insertion is at one of the moiety's ends—between a carbon or heteroatom and a hydrogen atom, in certain embodiments between a carbon and a hydrogen atom.

In case the conjugates of the present invention comprise one or more acidic or basic groups, the invention also comprises their corresponding pharmaceutically or toxicologically acceptable salts, in particular their pharmaceutically utilizable salts. Thus, the conjugates of the present invention comprising acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids, or quaternary ammoniums, such as tetrabutylammonium and cetyl trimethylammonium. Conjugates of the present invention comprising one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, trifluoroacetic acid and other acids known to the person skilled in the art. For the person skilled in the art further methods are known for converting the basic group into a cation like the alkylation of an amine group resulting in a positively-charged ammonium group and an appropriate counterion of the salt. If the conjugates of the present invention simultaneously comprise acidic and basic groups, the invention also includes, in addition to the salt forms mentioned, inner salts or betaines (zwitterions). The respective salts can be obtained by customary methods, which are known to the person skilled in the art like, for example by contacting these prodrugs with an organic or inorganic acid or base in a solvent or dispersant, or by anion exchange or cation exchange with other salts. The present invention also includes all salts of the conjugates of the present invention which, owing to low physiological compatibility, are not directly suitable for use in pharmaceuticals but which can be used, for example, as intermediates for chemical reactions or for the preparation of pharmaceutically acceptable salts.

As used herein, the term “pharmaceutically acceptable” means a substance that does not cause harm when administered to a patient and preferably means approved by a regulatory agency, such as the EMA (Europe) and/or the FDA (US) and/or any other national regulatory agency for use in animals, preferably for use in humans.

As used herein, the term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “small molecule drug” refers to drugs that are organic compounds with a molecular weight of less than 1000 Da, such as less than 900 Da or less than 800 Da.

As used herein, the term “medium molecule drug” refers to drugs that are organic compounds which are not peptides, and which are not proteins, and have a molecular weight ranging from and including 1 kDa to 7.5 kDa.

As used herein, the term “oligonucleotide” refers to a nucleic acid polymer of up to 100 bases and may be both DNA and RNA. The term also includes aptamers and morpholinos.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. The monomers may be identical, in which case the polymer is a homopolymer, or may be different, in which case the polymer is a heteropolymer. A heteropolymer may also be referred to as a “copolymer” and includes for example alternating copolymers in which monomers of different types alternate; periodic copolymers in which monomers of different types of monomers are arranged in a repeating sequence; statistical copolymers in which monomers of different types are arranged randomly; block copolymers in which blocks of different homopolymers consisting of only one type of monomers are linked by a covalent bond; and gradient copolymers in which the composition of different monomers changes gradually along a polymer chain. It is understood that a polymer may also comprise one or more other moieties, such as, for example, one or more functional groups.

Likewise, it is understood that also a peptide or protein is a polymer, even though the side chains of individual amino acid residues may be different. It is understood that for covalently crosslinked polymers, such as hydrogels, no meaningful molecular weight ranges can be provided.

As used herein, the term “polymeric” or “polymeric moiety” refers to a reagent or a moiety comprising one or more polymers or polymer moieties. A polymeric reagent or moiety may optionally also comprise one or more other moiety/moieties, which in certain embodiments are selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl and tetralinyl;
branching points, such as —CR<, >C< or —N<; and
linkages selected from the group comprising

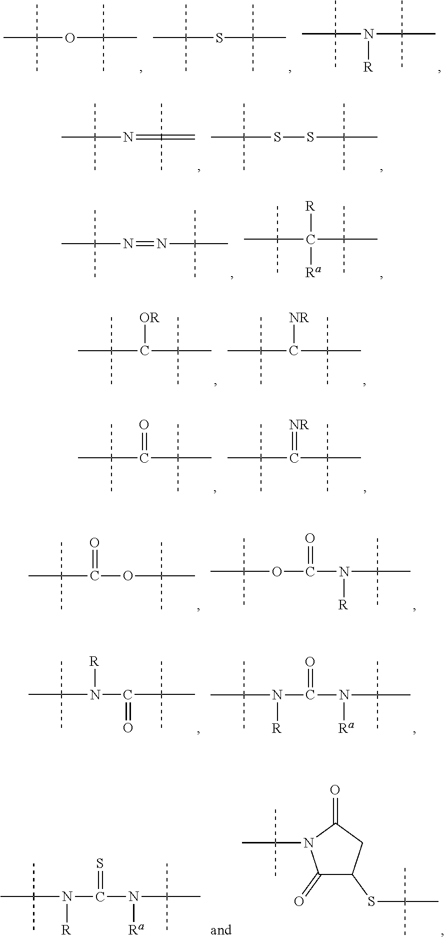

wherein
dashed lines indicate attachment to the remainder of the moiety or reagent;
—R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x+/−10%, in certain embodiments lies in a range of integers x+/−8%, in certain embodiments lies in a range of integers x+/−5% and in certain embodiments lies in a range of integers x+/−2%.

As used herein, the term “number average molecular weight” means the ordinary arithmetic mean of the molecular weights of the individual polymers.

As used herein, the term “PEG-based” in relation to a moiety or reagent means that said moiety or reagent comprises PEG. In certain embodiments, such PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60% (w/w) PEG, such as at least 70% (w/w) PEG, such as at least 80% (w/w) PEG, such as at least 90% (w/w) PEG, such as at least 95% (w/w) PEG. The remaining weight percentage of the PEG-based moiety or reagent may be other moieties, such as those selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl and tetralinyl;
branching points, such as —CR<, >C< or —N<; and
linkages selected from the group comprising

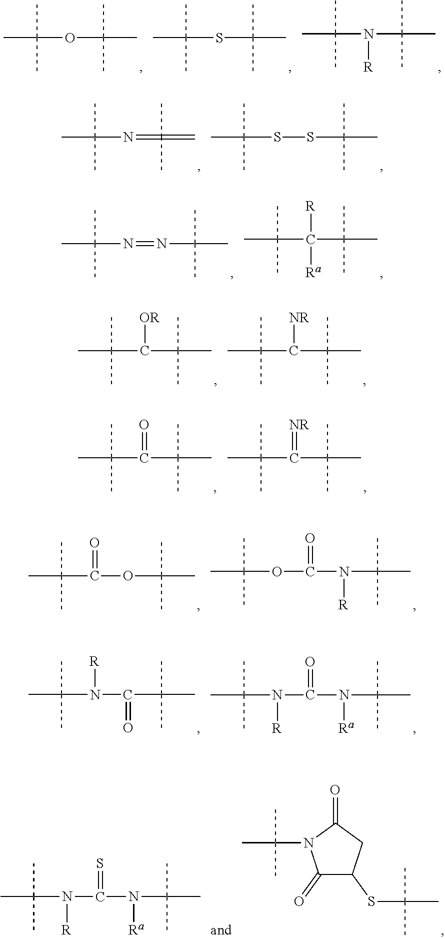

wherein
dashed lines indicate attachment to the remainder of the moiety or reagent; —R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “PEG-based comprising at least X % PEG” in relation to a moiety or reagent means that said moiety or reagent comprises at least X % (w/w) ethylene glycol units (—CH₂CH₂O—), wherein the ethylene glycol units may be arranged blockwise, alternating or may be randomly distributed within the moiety or reagent. In certain embodiments, all ethylene glycol units of said moiety or reagent are present in one block; the remaining weight percentage of the PEG-based moiety or reagent are other moieties in certain embodiments selected from the group consisting of
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group comprising

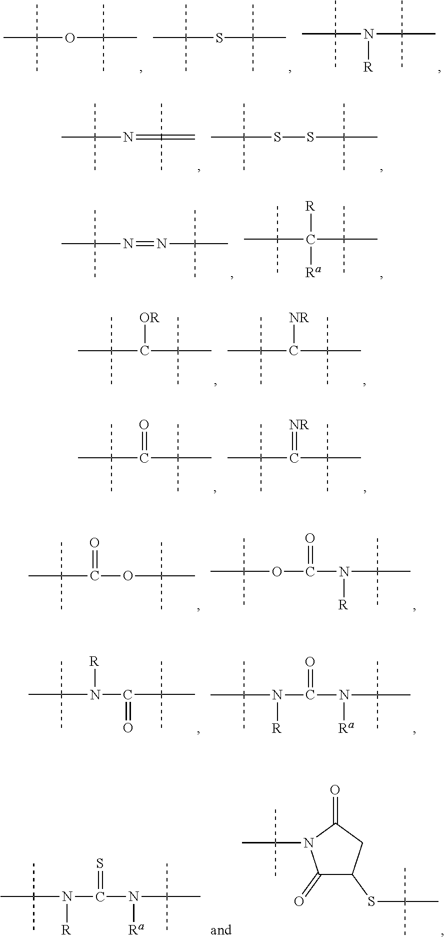

wherein
dashed lines indicate attachment to the remainder of the moiety or reagent, and wherein
—R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “hyaluronic acid-based” in relation to a moiety or reagent means that said moiety or reagent comprises hyaluronic acid. Such hyaluronic acid-based moiety or reagent comprises at least 10% (w/w) hyaluronic acid, such as at least 20% (w/w) hyaluronic acid, such as at least 30% (w/w) hyaluronic acid, such as at least 40% (w/w) hyaluronic acid, such as at least 50% (w/w) hyaluronic acid, such as at least 60% (w/w) hyaluronic acid, such as at least 70% (w/w) hyaluronic acid, such as at least 80% (w/w) hyaluronic acid, such as at least 90% (w/w) hyaluronic acid, or such as at least 95% (w/w) hyaluronic acid. The remaining weight percentage of the hyaluronic acid-based moiety or reagent may be other moieties, such as those selected from the group consisting of.
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl;
branching points, such as —CR<, >C< or —N<; and
linkages selected from the group consisting of

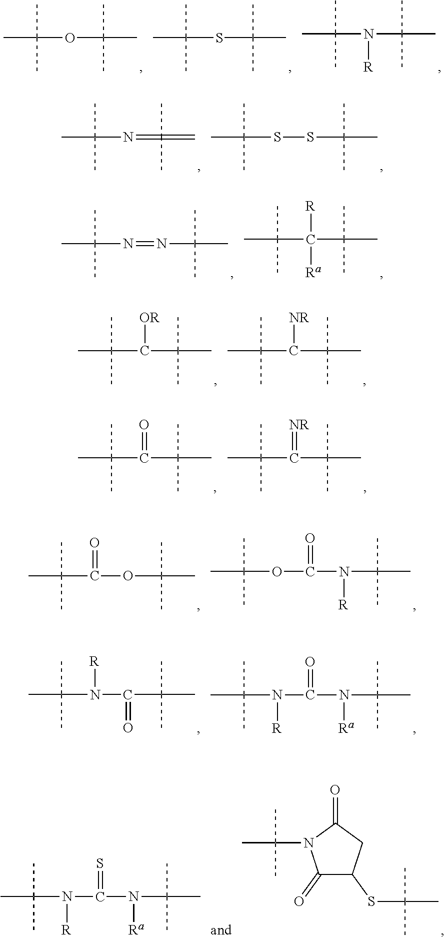

wherein
dashed lines indicate attachment to the remainder of the moiety or reagent;
—R and —R^{a} are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and which moieties and linkages are optionally further substituted.

As used herein, the term “hydrogel” means a hydrophilic or amphiphilic polymeric network composed of homopolymers or copolymers, which is insoluble due to the presence of hydrophobic interactions, hydrogen bonds, ionic interactions and/or covalent chemical crosslinks. The crosslinks provide the network structure and physical integrity.

As used herein, the term “random coil” refers to a peptide or protein adopting/having/forming, in certain embodiments having, a conformation which substantially lacks a defined secondary and tertiary structure as determined by circular dichroism spectroscopy performed in aqueous buffer at ambient temperature, and pH 7.4. In certain embodiments, the ambient temperature is about 20° C., i.e. between 18° C. and 22° C., while in certain embodiments the ambient temperature is 20° C.

As used herein, the term “spacer” or “spacer moiety” refers to a moiety suitable for connecting two moieties. Suitable spacers may be selected from the group consisting of C₁₋₅₀ alkyl, C₂₋₅₀ alkenyl and C₂₋₅₀ alkynyl, which C₁₋₅₀ alkyl, C₂₋₅₀ alkenyl or C₂₋₅₀ alkynyl is optionally interrupted by one or more groups selected from —NH—, —N(C₁₋₄ alkyl)-, —O—, —S—, —C(O)—, —C(O)NH—, —C(O)N(C₁₋₄ alkyl)-, —O—C(O)—, —S(O)—, —S(O)₂—, 4- to 7-membered heterocyclyl, phenyl and naphthyl and may optionally be substituted.

As used herein, the term “substituted” means that one or more —H atom(s) of a molecule or moiety are replaced by a different atom or a group of atoms, which are referred to as “substituent”.

As used herein, the term “substituent” refers in certain embodiments to a moiety selected from the group consisting of halogen, —CN, —C(O)OR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1a})(R^{x1b}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(Rxi)C(O)OR^{x1a}, —N(R^{x1})C(O)N(R^{x1a})(R^{x1b}), —OC(O)N(R^{x1})(R^{x1a}), -T⁰, C₁₋₅₀ alkyl, C₂₋₅₀ alkenyl and C₂₋₅₀ alkynyl; wherein -T⁰, C₁₋₅₀ alkyl, C₂₋₅₀ alkenyl and C₂₋₅₀ alkynyl are optionally substituted with one or more —R^{x2}, which are the same or different and wherein C₁₋₅₀ alkyl, C₂₋₅₀ alkenyl and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR³)(R^{x3a})—, —N(R^{x3})C(O)N(R^{x3a})— and —OC(O)N(R^{x3})—;

—R^{x1}, —R^{x1a}, —R^{x1b} are independently selected from the group consisting of —H, -T⁰, C₁₋₅₀ alkyl, C₂₋₅₀ alkenyl and C₂₋₅₀ alkynyl; wherein -T⁰, C₁₋₅₀ alkyl, C₂₋₅₀ alkenyl and C₂₋₅₀ alkynyl are optionally substituted with one or more —R^{x2}, which are the same or different and wherein C₁₋₅₀ alkyl, C₂₋₅₀ alkenyl and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR³)(R^{x3a})—, —N(R^{x3})C(O)N(R^{x3a})— and —OC(O)N(R^{x3})—;

each T⁰ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T⁰ is independently optionally substituted with one or more —R^{x2} which are the same or different;

each —R^{x2} is independently selected from the group consisting of halogen, —CN, oxo (═O), —C(O)OR^{x4}, —OR^{x4}, —C(O)R^{x4}, —C(O)N(R^{x4})(R^{x4a}), —S(O)₂N(R^{x4})(R^{x4a}), —S(O)N(R^{x4})(R^{x4a}), —S(O)₂R^{x4}, —S(O)R^{x4}, —N(R^{x4})S(O)₂N(R^{x4a})(R^{x4b}), —SR^{x4}, —N(R^{x4})(R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a})(R^{x4b}), —OC(O)N(R^{x4})(R^{x4a}) and C₁₋₆ alkyl; wherein C₁₋₆ alkyl is optionally substituted with one or more halogen, which are the same or different;

each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments, the term “substituent” refers to a moiety selected from the group consisting of halogen, halogen, —CN, —COOR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1})(R^{x1a}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1})(R^{x1a}), —OC(O)N(R^{x1})(R^{x1a}), -T⁰, C₁₋₁₀ alkyl, C₂₋₁₀ alkenyl, and C₂₋₁₀ alkynyl; wherein -T⁰, C₁₋₁₀ alkyl, C₂₋₁₀ alkenyl, and C₂₋₁₀ alkynyl are optionally substituted with one or more —R^{x2}, which are the same or different and wherein C₁₋₁₀ alkyl, C₂₋₁₀ alkenyl, and C₂₋₁₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR³)(R^{x3a})—, —N(R^{x3})C(O)N(R^{x3a})—, and —OC(O)N(R^{x3})—;

each —R^{x1}, —R^{x1a}, —R^{x1b}, —R^{x3}, —R^{x3a} is independently selected from the group consisting of —H, halogen, C₁₋₆ alkyl, C₂₋₆ alkenyl, and C₂₋₆ alkynyl;

each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different;

each —R^{x2} is independently selected from the group consisting of halogen, —CN, oxo (═O), —C(O)OR^{x4}, —OR^{x4}, —C(O)R^{x4}, —C(O)N(R^{x4})(R^{x4a}), —S(O)₂N(R^{x4})(R^{x4a}), —S(O)N(R^{x4})(R^{x4a}), —S(O)₂R^{x4}, —S(O)R^{x4}, —N(R^{x4})S(O)₂N(R^{x4a})(R^{x4b}), —SR^{x4}, —N(R^{x4})(R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{x4})C(O)N(R^{x4a})(R^{x4b}), —OC(O)N(R^{x4})(R^{x4a}) and C₁₋₆ alkyl; wherein C₁₋₆ alkyl is optionally substituted with one or more halogen, which are the same or different;

each —R^{x4}, —R^{x4a}, —R^{x4b} is independently selected from the group consisting of —H, halogen, C₁₋₆ alkyl, C₂₋₆ alkenyl, and C₂₋₆ alkynyl.

In certain embodiments, the term “substituent” refers to a moiety selected from the group consisting of halogen, —CN, —COOR^{x1}, —OR^{x1}, —C(O)R^{x1}, —C(O)N(R^{x1})(R^{x1a}), —S(O)₂N(R^{x1})(R^{x1a}), —S(O)N(R^{x1})(R^{x1a}), —S(O)₂R^{x1}, —S(O)R^{x1}, —N(R^{x1})S(O)₂N(R^{x1a})(R^{x1b}), —SR^{x1}, —N(R^{x1})(R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a})(R^{x1b}), —OC(O)N(R^{x1})(R^{x1a}), -T⁰, C₁₋₆ alkyl, C₂₋₆ alkenyl, and C₂₋₆ alkynyl; wherein -T⁰, C₁₋₆ alkyl, C₂₋₆ alkenyl, and C₂₋₆ alkynyl are optionally substituted with one or more —R^{x2}, which are the same or different and wherein C₁₋₆ alkyl, C₂₋₆ alkenyl, and C₂₋₆ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;

each —R^{x1}, —R^{x1a}, —R^{x1b}, —R^{x2}, —R^{x3}, —R^{x3a} is independently selected from the group consisting of —H, halogen, C₁₋₆ alkyl, C₂₋₆ alkenyl, and C₂₋₆ alkynyl;

each T⁰ is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T⁰ is independently optionally substituted with one or more —R^{x2} which are the same or different.

In certain embodiments, a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

As used herein, the term “therapeutically effective amount” means an amount sufficient to cure, alleviate or partially arrest the clinical manifestations of a given disease and its complications. Effective amounts for each purpose will depend on the severity of the disease or injury as well as the weight and general state of the subject.

As used herein, the term “sustained-release” refers to the property of a compound, such as the conjugates of the present invention, to release a drug with a release half-life of at least 1 day.

As used herein, the term “water-insoluble” refers to a compound of which less than 1 g can be dissolved in one liter of water at 20° C. to form a homogeneous solution. Accordingly, the term “water-soluble” refers to a compound of which 1 g or more can be dissolved in one liter of water at 20° C. to form a homogeneous solution.

In general, the term “comprise(s)” or “comprising” also encompasses “consist(s) of” or “consisting of”.

It is understood that “N⁺” in the phrases “an electron-donating heteroaromatic Ni-comprising moiety” and “attachment to the N⁺ of -D⁺” refers to a positively charged nitrogen atom.

It is also understood that “N” in the phrase “an electron-donating heteroaromatic N” refers to a nitrogen atom.

It is understood that, in certain embodiments, -D⁺ may comprise both an electron-donating heteroaromatic N⁺ and a quaternary ammonium cation and analogously the corresponding D may comprise both an electron-donating heteroaromatic N and a tertiary amine. It is also clear that if D is conjugated to -L¹-, then -D⁺ and -L¹- form a quaternary ammonium cation, for which there may be a counter anion. Examples of counter anions include, but are not limited to, chloride, bromide, acetate, bicarbonate, sulfate, bisulfate, nitrate, carbonate, alkyl sulfonate, aryl sulfonate and phosphate.

In certain embodiments, all moieties -D⁺ of the conjugate are identical, i.e. have the same chemical structure. In such case all moieties -D⁺ of the conjugate derive from the same type of drug molecule. It is understood that this means that all moieties -D⁺ originate from the same parent drug, but that there may be molecular rearrangements that for example lead to the formation of different tautomeric forms.

In certain embodiments, the conjugate of the present invention comprises different moieties -D⁺, i.e. comprises moieties -D⁺ with different chemical structures. These different structures derive from different types of drug molecules. It is understood that this does not include certain molecular rearrangements that for example lead to the formation of different tautomeric forms, which however may also be present. In certain embodiments, the conjugate of the present invention comprises two different types of moieties -D⁺. In certain embodiments, the conjugate of the present invention comprises three different types of moieties -D⁺. In certain embodiments, the conjugate of the present invention comprises four different types of moieties -D⁺. In certain embodiments, the conjugate of the present invention comprises five different types of moieties -D⁺.

If the conjugates of the present invention comprise more than one type of -D⁺, all moieties -D⁺ may be conjugated to the same type of -L¹- or may be conjugated to different types of -L¹-, i.e. a first type of -D⁺ may be conjugated to a first type of -L¹-, a second type of -D⁺ may be conjugated to a second type of -L¹- and so on. Using different types of -L¹- may, in certain embodiments, allow different release kinetics for different types of -D⁺, such as for example a faster release for a first type of -D⁺, a medium release for a second type of -D⁺ and a slow release for a third type of -D⁺. Accordingly, in certain embodiments the conjugates of the present invention comprise one type of -L¹-. In certain embodiments, the conjugates of the present invention comprise two types of -L¹-. In certain embodiments, the conjugates of the present invention comprise three types of -L¹-. In certain embodiments, the conjugates of the present invention comprise four types of -L¹-.

In certain embodiments, the conjugates of the present invention comprise one type of -D⁺ and one type of -L¹-. In certain embodiments, the conjugates of the present invention comprise two types of -D⁺ and two types of -L¹-. In certain embodiments, the conjugates of the present invention comprise three types of -D⁺ and three types of -L¹-. In certain embodiments, the conjugates of the present invention comprise four types of -D⁺ and four types of -L¹-.

In certain embodiments, all moieties -L¹- of the conjugate have the same structure. In certain embodiments the conjugate comprises two or more different types of moiety -L¹-, such as for example two, three, four or five different types of moiety -L¹-. Such two or more different types of moiety -L¹- may be conjugated to the same or different type of -D⁺. Using different types of -L¹- allows releasing the same or different type of drug D from the conjugate of the present invention with different release half-lives, such as when combining a first group of moieties -L¹- with a short release half-life with a second group of moieties -L¹- with a long release half-life.

In certain embodiments, -D⁺ is selected from the group consisting of small molecule, medium molecule, oligonucleotide, peptide and protein drug moieties.

In certain embodiments, -D⁺ is a small molecule drug moiety.

In certain embodiments, -D⁺ is a medium molecule drug moiety.

In certain embodiments, -D⁺ is an oligonucleotide drug moiety.

In certain embodiments, -D⁺ is a peptide drug moiety.

In certain embodiments, -D⁺ is a protein drug moiety. In certain embodiments, such protein moiety is a monoclonal or polyclonal antibody or fragment or fusion thereof.

It is also understood that for peptide and protein drug moieties an electron-donating heteroaromatic N⁺-comprising moiety may be provided by amino acids, such as for example, histidine.

In certain embodiments, -D⁺ is selected from the group consisting of axitinib, lenvatinib, topotecan, topotecan hydrochloride, abiraterone, LB-100, IB-01212, ⁶⁸Ga-BNOTA-PRGD2, venetoclax, gadoteridol, EC-1456, gadobutrol, mibefradil, resminostat, itarnafloxin, SHR-1258, neratinib maleate, imatinib, irinotecan, irinotecan hydrochloride, gilteritinib, abemaciclib, vinflunine, doxycycline, E-7016, HM-30181AK, A-366, GSK-923295, NMS-P937, EM-015, SB-743921, CX-5461, TP-0903, TLK-58747, BGP-15, BGJ-398, ⁹⁰Y-edotreotide, dovitinib, numonafide, ON-123300, vintafolide, KX2391, gadopentetatedimeglumine, gedatolisib, tasidotin HCl, tasidotin, naltrexone, CFI-400945 fumarate, pictilisib, felotaxel, OTX-008, trabectedin, NO-saquinavir, ENMD-2076, BGB-102, THZ-1, TSR-011, ONC-201, ASP-3026, galactosylceramide-enhanced vinorelbine, vinorelbine, ¹¹¹In-RP-782, DSR-6434, vincristine sulfate, apitolisib, MDX-1203, CNX-1351, CT-1578, TG-02, luminespib, vindesine-CB-3717 conjugates, AMG511, AZD1152hQPA accurins, mepacrine, CGM-097, TR-100, BGB-324, CEP-37440, OTSSP-167, APR-246, indotecan, gadoterate meglumine, lucanthone, navitoclax, Samarium (¹⁵³Sm) lexidronam, DMDAPatA, OCT-1002, BAY-87-2243, CEP-33779, XL-388, voruciclib, UNBS-5162, noscapine, PHA-665752, pyrrolobenzodiazepines, folate-vindesine hydrazide conjugate, BRN-103, NSC-134754, AMP-53, PKI-402, tariquidar, CG-200745, VO-100, PRLX-93936, cenisertib, SSR-125329, CRD-401, SN-24771, balamapimod, BIBX-1382, KW-2152, clidamycin, SN-23490, L-000021649, U-74389G, JNJ-17029259, PAK-200, NCO-700, B-220, pazelliptine, R-116010, RC-3940-II, BIM-46068, MDL-73811, CHIR-200131, halitulin, flezelastine, cinchonine, BN-52207, ABT-546, NSC-639366, datelliptium chloride, LY-329146, XR-9051, RSU-1069, epelmycin A, PNU-144113, FCE-27726, NSC-357704, PD-171851, DZ-3358, Goe-7874, Ro-44-5912, MDL-103323, mofarotene, Ro-46-7864, RU-45144, NC-190, NSC-646958, NSC-606985, VA-033, GI-149893, BBR-2378, NSC-639365, vinfosiltine, SDZ-62-434, BCH-2051, RB-90745, ER-37328, LY-326315, AN-1006, CP-117227, R-teludipine, RB-90740, SYUIQ-05, tamoxifen, norfloxacin, ciprofloxacin, elomotecan, vincristine, azoacridone, atiprimod, pelitinib, cemadotin, tozasertib, dofequidar, metoclopramide, procaine hydrochloride, siramesine, lurtotecan, auristatin PYE, C-1305, manzamines, becatecarin, soblidotin, tiamulin, tamolarizine, pibrozelesin, ladirubicin, declopramide, dexniguldipine, conophylline, anhydrovinblastine, canertinib, omacetaxine mepesuccinate, zosuquidar, rimcazole, astemizole, retelliptine, spirogermanium hydrochloride, mitonafide, tridolgosir, gefitinib, topixantrone, elacridar, desmethyl desaminopateamine A, alvespimycin hydrochloride, WBZ-7, 5-44563, GSK-1070916, RPR-203360, EU-5346, AT-9283, E-7974, GTx-134, A-620223, tesetaxel, SU 11274, E-7107, NRC-2694, PHA-793887, HB-19, CEP-28122, A-928605, PF-3758309, ^{99m}Tc-RP-527, MLN-576, JWH-018, Debio-0931, Debio-0932, Debio-1143, haloperidol hydrochloride, PF-337210, ABT-737, folatetubulysin conjugates, CP-31398, AV-412, GSK-1838705A, ABT-839, AEW-541, YHO-13351, PD-115934, dolastatin-10, EHT-1864, DX-52-1, RTA-502, BMS-753493, PD-166285, ANG-1009, Hoe-33342, STX-1801, BIBF-1000, ZK-191703, VX-322, Ro-28-2653, TH-237A, JNK-401, TAS-103, S-16020-2, NK-611, TOP-008, cyclopropylfentanyl, ICRF-193, tubulysin, ecteinascidins, L-745631, brigatinib, ALK-IN-1, MK-2461, MP-470, E-7050, EMD-387008, EMD-1204831, dolasetron, desacetylvinblastinehydrazide, liblomycin, ritonavir, dolaphenine androstane SR-25989, ellipticine, obatoclax, obatoclax mesylate, cabozantinib, Debio-1347, OSI-906, PAN-90806, BMS-536924, MK-8033, ARQ-197, MP-470, SGX-523, JNJ38877605, MGCD-265, SAR-125844, E-7050, R-7050, INCB-028060, EMD-387008, EMD-1204831, LY-2801653, AMG-208, acitazanolast, ledazerol, N-Desmethyl rosuvastatin, asperilicin C, asperilicin D, liblomycin, tazanolast, erdafitinib, afatinib, erlotinib, linifanib, sapitinib, BGB324 (BGB 324, R 428, R428, bemcentinib), tivantinib, ibrutinib, acalabrutinib, binimetinib, selumetinib, pimasertib, ulixertinib, MK-8353, GDC-0994, SCH772984, adavosertib, crizotinib, copanlisib, IPI-549, idelalisib, galunisertib, sorafenib, sunitinib, pazopanib, defactinib, dabrafenib, vemurafenib, encorafenib, indoximod, BMS-986205, AZD4635, vipadenant, CPI-444, preladenant, AZD5069, SX-682, X4P-001, PF-4136309, maraviroc, entinostat, chidamide, imiquimod, resiquimod, 852A, MEDI-9197, cyclic di-GMP, cyclic GMP-AMP and ADU-S100.

In certain embodiments, -D⁺ is selected from the group consisting of axitinib, lenvatinib, topotecan, topotecan hydrochloride, abiraterone, LB-100, IB-01212, ⁶⁸Ga-BNOTA-PRGD2, venetoclax, gadoteridol, EC-1456, gadobutrol, mibefradil, resminostat, itarnafloxin, SHR-1258, neratinib maleate, imatinib, irinotecan, irinotecan hydrochloride, gilteritinib, abemaciclib, vinflunine, doxycycline, E-7016, HM-30181AK, A-366, GSK-923295, NMS-P937, EM-015, SB-743921, CX-5461, TP-0903, TLK-58747, BGP-15, BGJ-398, ⁹⁰Y-edotreotide, dovitinib, numonafide, ON-123300, vintafolide, KX2391, gadopentetatedimeglumine, gedatolisib, tasidotin HCl, tasidotin, naltrexone, CFI-400945 fumarate, pictilisib, felotaxel, OTX-008, trabectedin, NO-saquinavir, ENMD-2076, BGB-102, THZ-1, TSR-011, ONC-201, ASP-3026, galactosylceramide-enhanced vinorelbine, vinorelbine, ¹¹¹In-RP-782, DSR-6434, vincristine sulfate, apitolisib, MDX-1203, CNX-1351, CT-1578, TG-02, luminespib, vindesine-CB-3717 conjugates, AMG511, AZD1152hQPA accurins, mepacrine, CGM-097, TR-100, BGB-324, CEP-37440, OTSSP-167, APR-246, indotecan, gadoterate meglumine, lucanthone, navitoclax, Samarium (¹⁵³Sm) lexidronam, DMDAPatA, OCT-1002, BAY-87-2243, CEP-33779, XL-388, voruciclib, UNBS-5162, noscapine, PHA-665752, pyrrolobenzodiazepines, folate-vindesine hydrazide conjugate, BRN-103, NSC-134754, AMP-53, PKI-402, tariquidar, CG-200745, VO-100, PRLX-93936, cenisertib, SSR-125329, CRD-401, SN-24771, balamapimod, BIBX-1382, KW-2152, clidamycin, SN-23490, L-000021649, U-74389G, JNJ-17029259, PAK-200, NCO-700, B-220, pazelliptine, R-116010, RC-3940-II, BIM-46068, MDL-73811, CHIR-200131, halitulin, flezelastine, cinchonine, BN-52207, ABT-546, NSC-639366, datelliptium chloride, LY-329146, XR-9051, RSU-1069, epelmycin A, PNU-144113, FCE-27726, NSC-357704, PD-171851, DZ-3358, Goe-7874, Ro-44-5912, MDL-103323, mofarotene, Ro-46-7864, RU-45144, NC-190, NSC-646958, NSC-606985, VA-033, GI-149893, BBR-2378, NSC-639365, vinfosiltine, SDZ-62-434, BCH-2051, RB-90745, ER-37328, LY-326315, AN-1006, CP-117227, R-teludipine, RB-90740, SYUIQ-05, tamoxifen, norfloxacin, ciprofloxacin, elomotecan, vincristine, azoacridone, atiprimod, pelitinib, cemadotin, tozasertib, dofequidar, metoclopramide, procaine hydrochloride, siramesine, lurtotecan, auristatin PYE, C-1305, manzamines, becatecarin, soblidotin, tiamulin, tamolarizine, pibrozelesin, ladirubicin, declopramide, dexniguldipine, conophylline, anhydrovinblastine, canertinib, omacetaxine mepesuccinate, zosuquidar, rimcazole, astemizole, retelliptine, spirogermanium hydrochloride, mitonafide, tridolgosir, gefitinib, topixantrone, elacridar, desmethyl desaminopateamine A, alvespimycin hydrochloride, WBZ-7, 5-44563, GSK-1070916, RPR-203360, EU-5346, AT-9283, E-7974, GTx-134, A-620223, tesetaxel, SU 11274, E-7107, NRC-2694, PHA-793887, HB-19, CEP-28122, A-928605, PF-3758309, ^{99m}Tc-RP-527, MLN-576, JWH-018, Debio-0931, Debio-0932, Debio-1143, haloperidol hydrochloride, PF-337210, ABT-737, folatetubulysin conjugates, CP-31398, AV-412, GSK-1838705A, ABT-839, AEW-541, YHO-13351, PD-115934, dolastatin-10, EHT-1864, DX-52-1, RTA-502, BMS-753493, PD-166285, ANG-1009, Hoe-33342, STX-1801, BIBF-1000, ZK-191703, VX-322, Ro-28-2653, TH-237A, JNK-401, TAS-103, S-16020-2, NK-611, TOP-008, cyclopropylfentanyl, ICRF-193, tubulysin, ecteinascidins, L-745631, brigatinib, ALK-IN-1, MK-2461, MP-470, E-7050, EMD-387008, EMD-1204831, dolasetron, desacetylvinblastinehydrazide, liblomycin, ritonavir, dolaphenine androstane SR-25989, ellipticine, obatoclax, obatoclax mesylate, cabozantinib, Debio-1347, OSI-906, PAN-90806, BMS-536924, MK-8033, ARQ-197, MP-470, SGX-523, JNJ38877605, MGCD-265, SAR-125844, E-7050, R-7050, INCB-028060, EMD-387008, EMD-1204831, LY-2801653, AMG-208, acitazanolast, ledazerol, N-Desmethyl rosuvastatin, asperilicin C, asperilicin D, liblomycin, tazanolast, erdafitinib, afatinib, erlotinib, linifanib, sapitinib, BGB324 (BGB 324, R 428, R428, bemcentinib), tivantinib, ibrutinib, acalabrutinib, binimetinib, selumetinib, pimasertib, ulixertinib, MK-8353, GDC-0994, SCH772984, adavosertib, crizotinib, copanlisib, IPI-549, idelalisib, galunisertib, sorafenib, sunitinib, pazopanib, defactinib, dabrafenib, vemurafenib, encorafenib, indoximod, BMS-986205, AZD4635, vipadenant, CPI-444, preladenant, AZD5069, SX-682, X4P-001, PF-4136309, maraviroc, entinostat, chidamide, imiquimod, resiquimod, 852A, MEDI-9197, cyclic di-GMP, cyclic GMP-AMP, ADU-S100 and milrinone.

In certain embodiments, -D⁺ is axitinib.

In certain embodiments, -D⁺ is lenvatinib.

In certain embodiments, -D⁺ is an electron-donating heteroaromatic N⁺-comprising moiety.

In certain embodiments, -D⁺ is an electron-donating heteroaromatic N⁺-comprising moiety selected from the group consisting of axitinib, lenvatinib, topotecan, topotecan hydrochloride, venetoclax, EC-1456, mibefradil, itamafloxin, SHR-1258, neratinib maleate, imatinib, irinotecan, irinotecan hydrochloride, gilteritinib, abemaciclib, abiraterone, E-7016, HM-30181AK, GSK-923295, NMS-P937, CX-5461, TP-0903, BGP-15, BGJ-398, dovitinib, ON-123300, vintafolide, KX2391, gedatolisib, CFI-400945 fumarate, pictilisib, OTX-008, NO-saquinavir, ENMD-2076, BGB-102, THZ-1, ASP-3026, DSR-6434, apitolisib, CNX-1351, CT-1578, TG-02, luminespib, vindesine-CB-3717 conjugates, AMG511, AZD1152hQPA accurins, mepacrine, TR-100, BGB-324, CEP-37440, OTSSP-167, navitoclax, DMDAPatA, BAY-87-2243, CEP-33779, XL-388, BRN-103, PKI-402, tariquidar, CG-200745, PRLX-93936, cenisertib, balamapimod, BIBX-1382, L-000021649, U-74389G, JNJ-17029259, ritonavir, B-220, pazelliptine, R-116010, RC-3940-II, MDL-73811, CHIR-200131, halitulin, flezelastine, cinchonine, dolaphenine androstane SR-25989, RSU-1069, PD-171851, DZ-3358, NC-190, NSC-606985, GI-149893, BBR-2378, RB-90745, ER-37328, CP-117227, RB-90740, SYUIQ-05, ellipticine, elomotecan, azoacridone, pelitinib, tozasertib, dofequidar, lurtotecan, C-1305, manzamines, canertinib, zosuquidar, astemizole, retelliptine, gefitinib, topixantrone, elacridar, desmethyl desaminopateamine A, WBZ-7, GSK-1070916, EU-5436, obatoclax, obatoclax mesylate, AT-9283, GTx-134, A-620223, tesetaxel, cabozantinib, NRC-2694, PHA-793887, HB-19, CEP-28122, A-928605, PF-3758309, ^{99m}Tc-RP-527, MLN-576, Debio-1347, Debio-0931, Debio-0932, PF-337210, folatetubulysin conjugates, CP-31398, AV-412, GSK-1838705A, AEW-541, PD-115934, dolastatin-10, EHT-1864, RTA-502, BMS-753493, PD-166285, Hoe-33342, OSI-906, PAN-90806, VX-322, JNK-401, TAS-103, S-16020-2, tubulysin, brigatinib, ALK-IN-1, BMS-536924, MK-8033, MK-2461, ARQ-197, MP-470, SGX-523, JNJ38877605, MGCD-265, SAR-125844, E-7050, R-7050, INCB-028060, EMD-387008, EMD-1204831, LY-2801653, AMG-208, acitazanolast, ledazerol, N-Desmethyl rosuvastatin, asperilicin C, asperilicin D, liblomycin, tazanolast, erdafitinib, afatinib, erlotinib, linifanib, sapitinib, BGB324 (BGB 324, R 428, R428, bemcentinib), ibrutinib, acalabrutinib, binimetinib, selumetinib, pimasertib, ulixertinib, MK-8353, GDC-0994, SCH772984, adavosertib, crizotinib, copanlisib, IPI-549, idelalisib, galunisertib, sorafenib, pazopanib, defactinib, dabrafenib, vemurafenib, encorafenib, BMS-986205, AZD4635, vipadenant, CPI-444, preladenant, AZD5069, SX-682, X4P-001, PF-4136309, maraviroc, entinostat, chidamide, imiquimod, resiquimod, 852A, MEDI-9197, cyclic di-GMP, cyclic GMP-AMP and ADU-S100.

In certain embodiments, -D⁺ is an electron-donating heteroaromatic N⁺-comprising moiety selected from the group consisting of axitinib, lenvatinib, topotecan, topotecan hydrochloride, venetoclax, EC-1456, mibefradil, itamafloxin, SHR-1258, neratinib maleate, imatinib, irinotecan, irinotecan hydrochloride, gilteritinib, abemaciclib, abiraterone, E-7016, HM-30181AK, GSK-923295, NMS-P937, CX-5461, TP-0903, BGP-15, BGJ-398, dovitinib, ON-123300, vintafolide, KX2391, gedatolisib, CFI-400945 fumarate, pictilisib, OTX-008, NO-saquinavir, ENMD-2076, BGB-102, THZ-1, ASP-3026, DSR-6434, apitolisib, CNX-1351, CT-1578, TG-02, luminespib, vindesine-CB-3717 conjugates, AMG511, AZD1152hQPA accurins, mepacrine, TR-100, BGB-324, CEP-37440, OTSSP-167, navitoclax, DMDAPatA, BAY-87-2243, CEP-33779, XL-388, BRN-103, PKI-402, tariquidar, CG-200745, PRLX-93936, cenisertib, balamapimod, BIBX-1382, L-000021649, U-74389G, JNJ-17029259, ritonavir, B-220, pazelliptine, R-116010, RC-3940-II, MDL-73811, CHIR-200131, halitulin, flezelastine, cinchonine, dolaphenine androstane SR-25989, RSU-1069, PD-171851, DZ-3358, NC-190, NSC-606985, GI-149893, BBR-2378, RB-90745, ER-37328, CP-117227, RB-90740, SYUIQ-05, ellipticine, elomotecan, azoacridone, pelitinib, tozasertib, dofequidar, lurtotecan, C-1305, manzamines, canertinib, zosuquidar, astemizole, retelliptine, gefitinib, topixantrone, elacridar, desmethyl desaminopateamine A, WBZ-7, GSK-1070916, EU-5436, obatoclax, obatoclax mesylate, AT-9283, GTx-134, A-620223, tesetaxel, cabozantinib, NRC-2694, PHA-793887, HB-19, CEP-28122, A-928605, PF-3758309, ^{99m}Tc-RP-527, MLN-576, Debio-1347, Debio-0931, Debio-0932, PF-337210, folatetubulysin conjugates, CP-31398, AV-412, GSK-1838705A, AEW-541, PD-115934, dolastatin-10, EHT-1864, RTA-502, BMS-753493, PD-166285, Hoe-33342, OSI-906, PAN-90806, VX-322, JNK-401, TAS-103, 5-16020-2, tubulysin, brigatinib, ALK-IN-1, BMS-536924, MK-8033, MK-2461, ARQ-197, MP-470, SGX-523, JNJ38877605, MGCD-265, SAR-125844, E-7050, R-7050, INCB-028060, EMD-387008, EMD-1204831, LY-2801653, AMG-208, acitazanolast, ledazerol, N-Desmethyl rosuvastatin, asperilicin C, asperilicin D, liblomycin, tazanolast, erdafitinib, afatinib, erlotinib, linifanib, sapitinib, BGB324 (BGB 324, R 428, R428, bemcentinib), ibrutinib, acalabrutinib, binimetinib, selumetinib, pimasertib, ulixertinib, MK-8353, GDC-0994, SCH772984, adavosertib, crizotinib, copanlisib, IPI-549, idelalisib, galunisertib, sorafenib, pazopanib, defactinib, dabrafenib, vemurafenib, encorafenib, BMS-986205, AZD4635, vipadenant, CPI-444, preladenant, AZD5069, SX-682, X4P-001, PF-4136309, maraviroc, entinostat, chidamide, imiquimod, resiquimod, 852A, MEDI-9197, cyclic di-GMP, cyclic GMP-AMP, ADU-S100 and milrinone.

In certain embodiments, -D⁺ is a quaternary ammonium cation comprising moiety.

In certain embodiments, -D⁺ is a quaternary ammonium cation comprising moiety selected from the group consisting of topotecan, topotecan hydrochloride, LB-100, IB-01212, ⁶⁸Ga-BNOTA-PRGD2, venetoclax, gadoteridol, EC-1456, gadobutrol, mibefradil, resminostat, itamafloxin, SHR-1258, neratinib maleate, imatinib, irinotecan, irinotecan hydrochloride, gilteritinib, abemaciclib, vinflunine, doxycycline, E-7016, HM-30181AK, A-366, GSK-923295, NMS-P937, EM-015, SB-743921, CX-5461, TP-0903, TLK-58747, BGP-15, BGJ-398, ⁹⁰Y-edotreotide, dovitinib, numonafide, ON-123300, vintafolide, KX2391, gadopentetatedimeglumine, gedatolisib, tasidotin HCl, tasidotin, naltrexone, CFI-400945 fumarate, pictilisib, felotaxel, OTX-008, trabectedin, NO-saquinavir, ENMD-2076, BGB-102, THZ-1, TSR-011, ONC-201, ASP-3026, galactosylceramide-enhanced vinorelbine, vinorelbine, ¹¹¹In-RP-782, DSR-6434, vincristine sulfate, apitolisib, MDX-1203, CNX-1351, CT-1578, TG-02, luminespib, vindesine-CB-3717 conjugates, AMG511, AZD1152hQPA accurins, mepacrine, CGM-097, TR-100, BGB-324, CEP-37440, OTSSP-167, APR-246, indotecan, gadoterate meglumine, lucanthone, navitoclax, Samarium (¹⁵³Sm) lexidronam, DMDAPatA, OCT-1002, BAY-87-2243, CEP-33779, XL-388, voruciclib, UNBS-5162, noscapine, PHA-665752, pyrrolobenzodiazepines, folate-vindesine hydrazide conjugate, BRN-103, NSC-134754, AMP-53, PKI-402, tariquidar, CG-200745, VO-100, PRLX-93936, cenisertib, SSR-125329, CRD-401, SN-24771, balamapimod, BIBX-1382, KW-2152, clidamycin, SN-23490, L-000021649, U-74389G, JNJ-17029259, PAK-200, NCO-700, B-220, pazelliptine, R-116010, RC-3940-II, BIM-46068, MDL-73811, CHIR-200131, halitulin, flezelastine, cinchonine, BN-52207, ABT-546, NSC-639366, datelliptium chloride, LY-329146, XR-9051, RSU-1069, epelmycin A, PNU-144113, FCE-27726, NSC-357704, PD-171851, DZ-3358, Goe-7874, Ro-44-5912, MDL-103323, mofarotene, Ro-46-7864, RU-45144, NC-190, NSC-646958, NSC-606985, VA-033, GI-149893, BBR-2378, NSC-639365, vinfosiltine, SDZ-62-434, BCH-2051, RB-90745, ER-37328, LY-326315, AN-1006, CP-117227, R-teludipine, RB-90740, SYUIQ-05, tamoxifen, norfloxacin, ciprofloxacin, elomotecan, vincristine, azoacridone, atiprimod, pelitinib, cemadotin, tozasertib, dofequidar, metoclopramide, procaine hydrochloride, siramesine, lurtotecan, auristatin PYE, C-1305, manzamines, becatecarin, soblidotin, tiamulin, tamolarizine, pibrozelesin, ladirubicin, declopramide, dexniguldipine, conophylline, anhydrovinblastine, canertinib, omacetaxine mepesuccinate, zosuquidar, rimcazole, astemizole, retelliptine, spirogermanium hydrochloride, mitonafide, tridolgosir, gefitinib, topixantrone, elacridar, desmethyl desaminopateamine A, alvespimycin hydrochloride, WBZ-7, S-44563, GSK-1070916, RPR-203360, EU-5346, AT-9283, E-7974, GTx-134, A-620223, tesetaxel, SU 11274, E-7107, NRC-2694, PHA-793887, HB-19, CEP-28122, A-928605, PF-3758309, ^{99m}Tc-RP-527, MLN-576, JWH-018, Debio-0931, Debio-0932, Debio-1143, haloperidol hydrochloride, PF-337210, ABT-737, folatetubulysin conjugates, CP-31398, AV-412, GSK-1838705A, ABT-839, AEW-541, YHO-13351, PD-115934, dolastatin-10, EHT-1864, DX-52-1, RTA-502, BMS-753493, PD-166285, ANG-1009, Hoe-33342, STX-1801, BIBF-1000, ZK-191703, VX-322, Ro-28-2653, TH-237A, JNK-401, TAS-103, S-16020-2, NK-611, TOP-008, cyclopropylfentanyl, ICRF-193, tubulysin, ecteinascidins, L-745631, brigatinib, ALK-IN-1, MK-2461, MP-470, E-7050, EMD-387008, EMD-1204831, dolasetron, desacetylvinblastinehydrazide, liblomycin, erdafitinib, afatinib, sapitinib, BGB324 (BGB 324, R 428, R428, bemcentinib), tivantinib, ibrutinib, MK-8353, SCH772984, adavosertib, copanlisib, sunitinib, pazopanib, indoximod, preladenant and X4P-001.

In certain embodiments, t of formula (I) is 0. In certain embodiments, t of formula (I) is 1. In certain embodiments, t of formula (I) is 2. In certain embodiments, t of formula (I) is 3. In certain embodiments, t of formula (I) is 4. In certain embodiments, t of formula (I) is 5. In certain embodiments, t of formula (I) is 6.

In certain embodiments, -A- is a ring selected from the group consisting of monocyclic or bicyclic aryl and heteroaryl, provided that -A- is connected to —Y and —C(R¹)(R^{1a})— via carbon atoms. In certain embodiments, -A- is substituted with one or more —R² which are the same or different. In certain embodiments, -A- is not substituted with —R².

In certain embodiments, -A- is selected from the group consisting of:

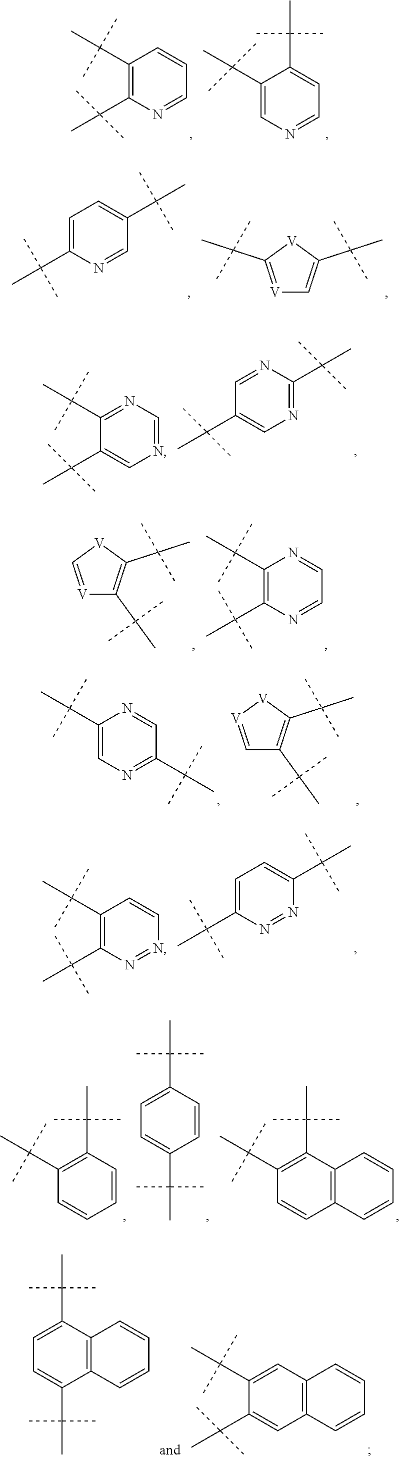

wherein each V is independently selected from the group consisting of O, S and N.

In certain embodiments, —R¹, —R^{1a} and each —R² are independently selected from the group consisting of —H, —C(O)OH, -halogen, —CN, —NO₂, —OH, C₁₋₆ alkyl, C₂₋₆ alkenyl and C₂₋₆ alkynyl.

In certain embodiments, —R¹, —R^{1a} and each —R² are independently selected from the group consisting of —H, —C(O)OH, —CN, C₁₋₆ alkyl, C₂₋₆ alkenyl and C₂₋₆ alkynyl.

In certain embodiments, —R¹ is —H. In certain embodiments, —R¹ is —C(O)OH. In certain embodiments, —R¹ is -halogen. In certain embodiments, —R¹ is —F. In certain embodiments, —R¹ is —CN. In certain embodiments, —R¹ is —NO₂. In certain embodiments, —R¹ is —OH. In certain embodiments, —R¹ is C₁₋₆ alkyl. In certain embodiments, —R¹ is C₂₋₆ alkenyl. In certain embodiments, —R¹ is C₂₋₆ alkynyl.

In certain embodiments, —R^{1a} is —H. In certain embodiments, —R^{1a} is —C(O)OH. In certain embodiments, —R^{1a} is -halogen. In certain embodiments, —R^{1a} is —F. In certain embodiments, —R^{1a} is —CN. In certain embodiments, —R^{1a} is —NO₂. In certain embodiments, —R^{1a} is —OH. In certain embodiments, —R^{1a} is C₁₋₆ alkyl. In certain embodiments, —R^{1a} is C₂₋₆ alkenyl. In certain embodiments, —R^{1a} is C₂₋₆ alkynyl.

In certain embodiments, each of —R² is independently selected from the group consisting of —H, —C(O)OH, -halogen, —CN, —NO₂, —OH, C₁₋₆ alkyl, C₂₋₆ alkenyl and C₂₋₆ alkynyl. In certain embodiments, each of —R² is —H. In certain embodiments, each of —R² is —C(O)OH. In certain embodiments, each of —R² is -halogen. In certain embodiments, each of —R² is —F. In certain embodiments, each of —R² is —CN. In certain embodiments, each of —R² is —NO₂. In certain embodiments, each of —R² is —OH. In certain embodiments, each of —R² is C₁₋₆ alkyl. In certain embodiments, each of —R² is C₂₋₆ alkenyl. In certain embodiments, each of —R² is C₂₋₆ alkynyl.

In certain embodiments, T is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, T is phenyl. In certain embodiments, T is naphthyl. In certain embodiments, T is indenyl. In certain embodiments, T is indanyl. In certain embodiments, T is tetralinyl. In certain embodiments, T is C₃₋₁₀ cycloalkyl. In certain embodiments, T is 3- to 10-membered heterocyclyl. In certain embodiments, T is 8- to 11-membered heterobicyclyl.

In certain embodiments, T is substituted with one or more —R³, which are the same or different. In certain embodiments, T is substituted with one —R³. In certain embodiments, T is not substituted with —R³.

In certain embodiments, —R³ is selected from the group consisting of —H, —NO₂, —OCH₃, —CN, —N(R⁴)(R^{4a}), —OH, —C(O)OH and C₁₋₆ alkyl.

In certain embodiments, —R³ is —H. In certain embodiments, —R³ is —NO₂. In certain embodiments, —R³ is —OCH₃. In certain embodiments, —R⁵ is —CN. In certain embodiments, —R³ is —N(R⁴)(R^{4a}). In certain embodiments, —R³ is —OH. In certain embodiments, —R³ is —C(O)OH. In certain embodiments, —R³ is C₁₋₆ alkyl.

In certain embodiments, —R⁴ and —R^{4a} are independently selected from the group consisting of —H and C₁₋₆ alkyl. In certain embodiments, —R⁴ is —H. In certain embodiments, —R⁴ is C₁₋₆ alkyl. In certain embodiments, —R^{4a} is —H. In certain embodiments, —R^{4a} is C₁₋₆ alkyl.

In certain embodiments, —Y is selected from the group consisting of

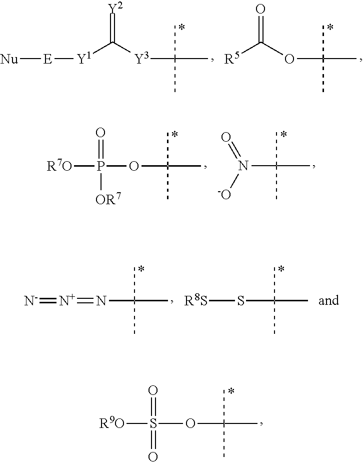

wherein —Nu, -E-, —Y¹—, ═Y², —Y³—, —R⁵, —R⁷, —R⁸ and —R⁹ are defined as above.

In certain embodiments, —Y is

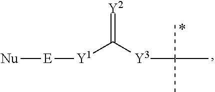

wherein —Nu, -E, —Y¹—, ═Y² and —Y³— are as defined above and the dashed line marked with an asterisk indicates the attachment to -A-. It is understood that in this instance the release of the drug D is not triggered by an enzyme, and that the drug is released in its unmodified, pharmacologically fully active form in the absence of an enzyme.

In certain embodiments, —Nu is a nucleophile selected from the group consisting of primary, secondary, or tertiary amine and amide. In certain embodiments, —Nu is a primary amine. In certain embodiments, —Nu is a secondary amine. In certain embodiments, —Nu is a tertiary amine. In certain embodiments, —Nu is an amide.

In certain embodiments, —Y¹— is selected from the group consisting of —O—, —C(R¹⁰)(R^{10a})—, —N(R¹¹)— and —S—. In certain embodiments, —Y¹— is —O—. In certain embodiments, —Y¹— is —C(R¹⁰)(R^{10a})—. In certain embodiments, —Y¹— is —N(R¹¹)—. In certain embodiments, —Y¹— is —S—.

In certain embodiments, ═Y² is selected from the group consisting of ═O, ═S and ═N(R¹²). In certain embodiments, ═Y² is ═O. In certain embodiments, ═Y² is ═S. In certain embodiments, ═Y² is ═N(R¹²).

In certain embodiments, —Y³— is selected from the group consisting of —O—, —S— and —N(R¹³)—. In certain embodiments, —Y³— is —O—. In certain embodiments, —Y³— is —S—. In certain embodiments, —Y³— is —N(R¹³)—.

In certain embodiments, —Y¹— is —N(R¹¹)—, ═Y² is ═O and —Y³— is —O—.

In certain embodiments, —Y¹— is —N(R¹¹)—, ═Y² is ═O, —Y³— is —O— and —Nu is —N(CH₃)₂.

In certain embodiments, -E- is selected from the group consisting of C₁₋₆ alkyl, C₂₋₆ alkenyl, C₂₋₆ alkynyl and -Q-. In certain embodiments, -E- is C₁₋₆ alkyl. In certain embodiments, -E- is C₂₋₆ alkenyl. In certain embodiments, -E- is C₂₋₆ alkynyl. In certain embodiments, -E- is -Q-.

In certain embodiments, Q is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, Q is phenyl. In certain embodiments, Q is naphthyl. In certain embodiments, Q is indenyl. In certain embodiments, Q is indanyl. In certain embodiments, Q is tetralinyl. In certain embodiments, Q is C₃₋₁₀ cycloalkyl. In certain embodiments, Q is 3- to 10-membered heterocyclyl. In certain embodiments, Q is 8- to 11-membered heterobicyclyl. In certain embodiments, Q is substituted with one or more —R¹⁴. In certain embodiments, Q is not substituted with —R¹⁴.

In certain embodiments, —R⁵, —R⁶, each —R⁷, —R⁸, —R⁹, —R¹⁰, —R^{10a}, —R¹¹, —R¹² and —R¹³ are independently selected from the group consisting of C₁₋₂₀ alkyl, C₂₋₂₀ alkenyl, C₂₋₂₀ alkynyl and -Q.

In certain embodiments, —R⁵ is C₁₋₂₀ alkyl. In certain embodiments, —R⁵ is C₂₋₂₀ alkenyl. In certain embodiments, —R⁵ is C₂₋₂₀ alkynyl. In certain embodiments, —R⁵ is -Q.

In certain embodiments, —R⁶ is C₁₋₂₀ alkyl. In certain embodiments, —R⁶ is C₂₋₂₀ alkenyl. In certain embodiments, —R⁶ is C₂₋₂₀ alkynyl. In certain embodiments, —R⁶ is -Q.

In certain embodiments, each of —R⁷ is independently selected from the group consisting of C₁₋₂₀ alkyl, C₂₋₂₀ alkenyl, C₂₋₂₀ alkynyl and -Q. In certain embodiments, each of —R⁷ is C₁₋₂₀ alkyl. In certain embodiments, each of —R⁷ is C₂₋₂₀ alkenyl. In certain embodiments, each of —R⁷ is C₂₋₂₀ alkynyl. In certain embodiments, each of —R⁷ is -Q.

In certain embodiments, —R⁸ is C₁₋₂₀ alkyl. In certain embodiments, —R⁸ is C₂₋₂₀ alkenyl. In certain embodiments, —R⁸ is C₂₋₂₀ alkynyl. In certain embodiments, —R⁸ is -Q.

In certain embodiments, —R⁹ is C₁₋₂₀ alkyl. In certain embodiments, —R⁹ is C₂₋₂₀ alkenyl. In certain embodiments, —R⁹ is C₂₋₂₀ alkynyl. In certain embodiments, —R⁹ is -Q.

In certain embodiments, —R¹⁰ is C₁₋₂₀ alkyl. In certain embodiments, —R¹⁰ is C₂₋₂₀ alkenyl. In certain embodiments, —R¹⁰ is C₂₋₂₀ alkynyl. In certain embodiments, —R¹⁰ is -Q.

In certain embodiments, —R^{10a} is C₁₋₂₀ alkyl. In certain embodiments, —R^{10a} is C₂₋₂₀ alkenyl. In certain embodiments, —R^{10a} is C₂₋₂₀ alkynyl. In certain embodiments, —R^{10a} is -Q.

In certain embodiments, —R¹¹ is C₁₋₂₀ alkyl. In certain embodiments, —R¹¹ is C₂₋₂₀ alkenyl. In certain embodiments, —R¹¹ is C₂₋₂₀ alkynyl. In certain embodiments, —R¹¹ is -Q.

In certain embodiments, —R¹² is C₁₋₂₀ alkyl. In certain embodiments, —R¹² is C₂₋₂₀ alkenyl. In certain embodiments, —R¹² is C₂₋₂₀ alkynyl. In certain embodiments, —R¹² is -Q.

In certain embodiments, —R¹³ is C₁₋₂₀ alkyl. In certain embodiments, —R¹³ is C₂₋₂₀ alkenyl. In certain embodiments, —R¹³ is C₂₋₂₀ alkynyl. In certain embodiments, —R¹³ is -Q.

In certain embodiments, —R¹⁴, —R¹⁵ and —R^{15a} are selected from the group consisting of —H and C₁₋₆ alkyl.

In certain embodiments, —R¹⁴ is —H. In certain embodiments, —R¹⁴ is C₁₋₆ alkyl.

In certain embodiments, —R¹⁵ is —H. In certain embodiments, —R¹⁵ is C₁₋₆ alkyl. In certain embodiments, —R^{15a} is —H. In certain embodiments, —R^{15a} is C₁₋₆ alkyl.

In certain embodiments, —Y is

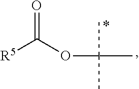

wherein —R⁵ is as defined above and the dashed line marked with an asterisk indicates the attachment to -A-.

In certain embodiments, —Y is

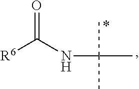

wherein —R⁶ is as defined above and the dashed line marked with an asterisk indicates the attachment to -A-.

In certain embodiments, —R⁶ is of formula (a):

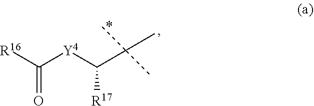

wherein —Y⁴— is selected from the group consisting of C₃₋₁₀ cycloalkyl, 3- to 10-membered heterocyclyl and 8- to 11-membered heterobicyclyl, which are optionally substituted with one or more —R¹⁸ which are the same or different; —R¹⁶ and —R¹⁷ are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R¹⁸ which are the same or different; and wherein C₁₋₁₀ alkyl, C₂₋₁₀ alkenyl and C₂₋₁₀ alkynyl are optionally interrupted by one or more groups selected from the group consisting of -A′-, —C(O)O—, —O—, —C(O)—, —C(O)N(R¹⁹)—, —S(O)₂N(R¹⁹), —S(O)N(R¹⁹)—, —S(O)₂—, —S(O)—, —N(R¹⁹)S(O)₂N(R^{19a})—, —S—, —N(R¹⁹)—, —OC(OR¹⁹)R^{19a}—, —N(R¹⁹)C(O)N(R^{19a})—, —OC(O)N(R¹⁹)— and —N(R¹⁹)C(NH₂)N(R^{19a})—;
   each A′ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each A′ is independently optionally substituted with one or more —R¹⁸ which are the same or different;
   wherein —R¹⁸, —R¹⁹ and —R^{19a} are independently selected from the group consisting of —H and C₁₋₆ alkyl; wherein C₁₋₆ alkyl is optionally substituted with one or more halogen, which are the same or different; and
wherein the dashed line marked with an asterisk indicates the attachment to the rest of —Y.

In certain embodiments, —Y⁴— is selected from the group consisting of C₃₋₁₀ cycloalkyl, 3- to 10-membered heterocyclyl and 8- to 11-membered heterobicyclyl. In certain embodiments, —Y⁴— is C₃₋₁₀ cycloalkyl. In certain embodiments, —Y⁴— is 3- to 10-membered heterocyclyl. In certain embodiments, —Y⁴— is 8- to 11-membered heterobicyclyl. In certain embodiments, —Y⁴— is substituted with one or more —R¹⁸ which are the same or different. In certain embodiments, —Y⁴— is not substituted with —R¹⁸.

In certain embodiments, —R¹⁶ and —R¹⁷ are selected from the group consisting of C₁₋₁₀ alkyl, C₂₋₁₀ alkenyl and C₂₋₁₀ alkynyl. In certain embodiments, —R¹⁶ is C₁₋₁₀ alkyl. In certain embodiments, —R¹⁶ is C₂₋₁₀ alkenyl. In certain embodiments, —R¹⁶ is C₂₋₁₀ alkynyl. In certain embodiments, —R¹⁷ is C₁₋₁₀ alkyl. In certain embodiments, —R¹⁷ is C₂₋₁₀ alkenyl. In certain embodiments, —R¹⁷ is C₂₋₁₀ alkynyl.

In certain embodiments, A′ is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, A′ is phenyl. In certain embodiments, A′ is naphthyl. In certain embodiments, A′ is indenyl. In certain embodiments, A′ is indanyl. In certain embodiments, A′ is tetralinyl. In certain embodiments, A′ is C₃₋₁₀ cycloalkyl. In certain embodiments, A′ is 3- to 10-membered heterocyclyl. In certain embodiments, A′ is 8- to 11-membered heterobicyclyl. In certain embodiments, A′ is substituted with one or more —R¹⁸, which are the same or different. In certain embodiments, A′ is not substituted with —R¹⁸.

In certain embodiments, —R¹⁸, —R¹⁹ and —R^{19a} are selected from the group consisting of —H and C₁₋₆ alkyl. In certain embodiments, —R¹⁸ is —H. In certain embodiments, —R¹⁸ is C₁₋₆ alkyl. In certain embodiments, —R¹⁹ is —H. In certain embodiments, —R¹⁹ is C₁₋₆ alkyl. In certain embodiments, —R^{19a} is —H. In certain embodiments, —R^{19a} is C₁₋₆ alkyl.

In certain embodiments, —R⁶ is of formula (b):

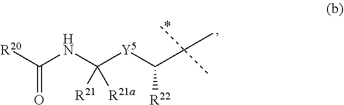

wherein —Y⁵— is selected from the group consisting of -Q′-, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²³, which are the same or different; and wherein C₁₋₁₀ alkyl, C₂₋₁₀ alkenyl and C₂₋₁₀ alkynyl are optionally interrupted by one or more groups selected from the group consisting of -Q′-, —C(O)O—, —O—, —C(O)—, —C(O)N(R²⁴)—, —S(O)₂N(R²⁴)—, —S(O)N(R²⁴)—, —S(O)₂—, —S(O)—, —N(R²⁴)S(O)₂N(R^{24a})—, —S—, —N(R²⁴)—, —OC(OR²⁴)R^{24a}—, —N(R²⁴)C(O)N(R^{24a})—, —OC(O)N(R²⁴)— and —N(R²⁴)C(NH₂)N(R^{24a})—;
—R²⁰, —R²¹, —R^{21a} and —R²² are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²³ which are the same or different; and wherein C₁₋₁₀ alkyl, C₂₋₁₀ alkenyl and C₂₋₁₀ alkynyl are optionally interrupted by one or more groups selected from the group consisting of -Q′-, —C(O)O—, —O—, —C(O)—, —C(O)N(R²⁴)—, —S(O)₂N(R²⁴)—, —S(O)N(R²⁴)—, —S(O)₂—, —S(O)—, —N(R²⁴)S(O)₂N(R^{24a})—, —S—, —N(R²⁴)—, —OC(OR²⁴)R^{24a}—, —N(R²⁴)C(O)N(R^{24a})—, —OC(O)N(R²⁴)— and —N(R²⁴)C(NH₂)N(R^{24a})—;
   each Q′ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q′ is independently optionally substituted with one or more —R²³, which are the same or different;
   wherein —R²³, —R²⁴ and —R^{24a} are independently selected from the group consisting of —H and C₁₋₆ alkyl; wherein C₁₋₆ alkyl is optionally substituted with one or more halogen, which are the same or different;
optionally, the pair —R²¹/—R^{21a} is joined together with the atoms to which is attached to form a C₃₋₁₀ cycloalkyl, 3- to 10-membered heterocyclyl or an 8- to 11-membered heterobicyclyl; and
wherein the dashed line marked with an asterisk indicates the attachment to the rest of —Y.

In certain embodiments, —Y⁵— is selected from the group consisting of -Q′-, C₁₋₁₀ alkyl, C₂₋₁₀ alkenyl and C₂₋₁₀ alkynyl. In certain embodiments, —Y⁵— is -Q′-. In certain embodiments, —Y⁵— is C₁₋₁₀ alkyl. In certain embodiments, —Y⁵— is C₂₋₁₀ alkenyl. In certain embodiments, —Y⁵— is C₂₋₁₀ alkynyl.

In certain embodiments, Q′ is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, Q′ is phenyl. In certain embodiments, Q′ is naphthyl. In certain embodiments, Q′ is indenyl. In certain embodiments, Q′ is indanyl. In certain embodiments, Q′ is C₃₋₁₀ cycloalkyl. In certain embodiments, Q′ is 3- to 10-membered heterocyclyl. In certain embodiments, Q′ is 8- to 11-membered heterobicyclyl. In certain embodiments, Q′ is substituted with one or more —R²³ which are the same or different. In certain embodiments, Q′ is not substituted with —R²³.

In certain embodiments, —R²⁰, —R²¹, —R^{21a} and —R²² are selected from the group consisting of —H, C₁₋₁₀ alkyl, C₂₋₁₀ alkenyl and C₂₋₁₀ alkynyl.

In certain embodiments, —R²⁰ is —H. In certain embodiments, —R²⁰ is C₁₋₁₀ alkyl. In certain embodiments, —R²⁰ is C₂₋₁₀ alkenyl. In certain embodiments, —R²⁰ is C₂₋₁₀ alkynyl.

In certain embodiments, —R²¹ is —H. In certain embodiments, —R²¹ is C₁₋₁₀ alkyl. In certain embodiments, —R²¹ is C₂₋₁₀ alkenyl. In certain embodiments, —R²¹ is C₂₋₁₀ alkynyl.

In certain embodiments, —R^{21a} is —H. In certain embodiments, —R^{21a} is C₁₋₁₀ alkyl. In certain embodiments, —R^{21a} is C₂₋₁₀ alkenyl. In certain embodiments, —R^{21a} is C₂₋₁₀ alkynyl.

In certain embodiments, —R²² is —H. In certain embodiments, —R²² is C₁₋₁₀ alkyl. In certain embodiments, —R²² is C₂₋₁₀ alkenyl. In certain embodiments, —R²² is C₂₋₁₀ alkynyl.

In certain embodiments, —R²³, —R²⁴ and —R^{24a} are selected from the group consisting of —H and C₁₋₆ alkyl.

In certain embodiments, —R²³ is —H. In certain embodiments, —R²³ is C₁₋₆ alkyl.

In certain embodiments, —R²⁴ is —H. In certain embodiments, —R²⁴ is C₁₋₆ alkyl.

In certain embodiments, —R^{24a} is —H. In certain embodiments, —R^{24a} is C₁₋₆ alkyl.

In certain embodiments, the pair —R²¹/—R^{21a} is joined together with the atoms to which is attached to form a C₃₋₁₀ cycloalkyl.

In certain embodiments, —R⁶ is of formula (c):

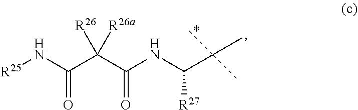

wherein
—R²⁵, —R²⁶, —R^{26a} and —R²⁷ are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²⁸ which are the same or different; and wherein C₁₋₁₀ alkyl, C₂₋₁₀ alkenyl and C₂₋₁₀ alkynyl are optionally interrupted by one or more groups selected from the group consisting of -Q*-, —C(O)O—, —O—, —C(O)—, —C(O)N(R²⁹)—, —S(O)₂N(R²⁹)—, —S(O)N(R²⁹)—, —S(O)₂—, —S(O)—, —N(R²⁹)S(O)₂N(R^{29a})—, —S—, —N(R²⁹)—, —OC(OR²⁹)R^{29a}—, —N(R²⁹)C(O)N(R^{29a})—, —OC(O)N(R²⁹)— and —N(R²⁹)C(NH₂)N(R^{29a})—;
   each Q* is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q* is independently optionally substituted with one or more —R²⁸, which are the same or different;
   wherein —R²⁸, —R²⁹ and —R^{29a} are independently selected from the group consisting of —H and C₁₋₆ alkyl; wherein C₁₋₆ alkyl is optionally substituted with one or more halogen, which are the same or different;
optionally, the pair —R²⁶/—R^{26a} is joined together with the atoms to which is attached to form a C₃₋₁₀ cycloalkyl, 3- to 10-membered heterocyclyl or an 8- to 11-membered heterobicyclyl; and
wherein the dashed line marked with an asterisk indicates the attachment to the rest of —Y.

In certain embodiments, —R²⁵, —R²⁶, —R^{26a} and —R²⁷ are selected from the group consisting of —H, C₁₋₁₀ alkyl, C₂₋₁₀ alkenyl and C₂₋₁₀ alkynyl.

In certain embodiments, —R²⁵ is —H. In certain embodiments, —R²⁵ is C₁₋₁₀ alkyl. In certain embodiments, —R²⁵ is C₂₋₁₀ alkenyl. In certain embodiments, —R²⁵ is C₂₋₁₀ alkynyl.

In certain embodiments, —R²⁶ is —H. In certain embodiments, —R²⁶ is C₁₋₁₀ alkyl. In certain embodiments, —R²⁶ is C₂₋₁₀ alkenyl. In certain embodiments, —R²⁶ is C₂₋₁₀ alkynyl.

In certain embodiments, —R^{26a} is —H. In certain embodiments, —R^{26a} is C₁₋₁₀ alkyl. In certain embodiments, —R^{26a} is C₂₋₁₀ alkenyl. In certain embodiments, —R^{26a} is C₂₋₁₀ alkynyl.

In certain embodiments, —R²⁷ is —H. In certain embodiments, —R²⁷ is C₁₋₁₀ alkyl. In certain embodiments, —R²⁷ is C₂₋₁₀ alkenyl. In certain embodiments, —R²⁷ is C₂₋₁₀ alkynyl.

In certain embodiments, Q* is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments, Q* is phenyl. In certain embodiments, Q* is naphthyl. In certain embodiments, Q* is indenyl. In certain embodiments, Q* is indanyl. In certain embodiments, Q* is tetralinyl. In certain embodiments, Q* is C₃₋₁₀ cycloalkyl. In certain embodiments, Q* is 3- to 10-membered heterocyclyl. In certain embodiments, Q* is 8- to 11-membered heterobicyclyl. In certain embodiments, Q* is substituted with one or more —R²⁸, which are the same or different. In certain embodiments, Q* is not substituted with —R²⁸.

In certain embodiments, —R²⁸, —R²⁹ and —R^{29a} are selected from the group consisting of —H and C₁₋₆ alkyl. In certain embodiments, —R²⁸ is —H. In certain embodiments, —R²⁸ is C₁₋₆ alkyl. In certain embodiments, —R²⁹ is —H. In certain embodiments, —R²⁹ is C₁₋₆ alkyl. In certain embodiments, —R^{29a} is —H. In certain embodiments, —R^{29a} is C₁₋₆ alkyl.

In certain embodiments, the pair —R²⁶/—R^{26a} is joined together with the atoms to which is attached to form a C₃₋₁₀ cycloalkyl.

In certain embodiments, the pair —R²⁶/—R^{26a} is joined together with the atoms to which is attached to form a cyclobutyl.

In certain embodiments, —Y is

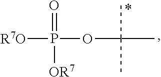

wherein each —R⁷ is as defined above and the dashed line marked with an asterisk indicates the attachment to -A-. It is understood that in this instance the release of the drug D may be triggered by an enzyme, such as phosphatase.

In certain embodiments, —Y is

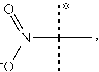

wherein the dashed line marked with an asterisk indicates the attachment to -A-.

In certain embodiments, —Y is

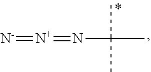

wherein the dashed line marked with an asterisk indicates the attachment to -A-.

In certain embodiments, —Y is

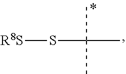

wherein —R⁸ is as defined above and the dashed line marked with an asterisk indicates the attachment to -A-.

In certain embodiments, —Y is

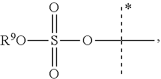

wherein —R⁹ is as defined above and the dashed line marked with an asterisk indicates the attachment to -A-. It is understood that in this instance the release of the drug D may be triggered by an enzyme, such as sulfatase.

In certain embodiments, —Y is

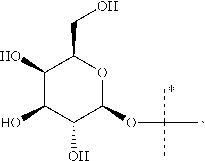

wherein the dashed line marked with an asterisk indicates the attachment to -A-. It is understood that in this instance the release of the drug D may be triggered by an enzyme, such as α-galactosidase.

In certain embodiments, —Y is

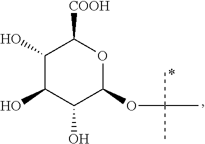

wherein the dashed line marked with an asterisk indicates the attachment to -A-. It is understood that in this instance the release of the drug D may be triggered by an enzyme, such as β-glucuronidase.

In certain embodiments, —Y is

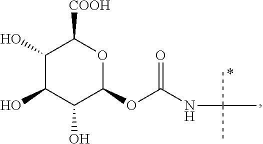

wherein the dashed line marked with an asterisk indicates the attachment to -A-. It is understood that in this instance the release of the drug D may be triggered by an enzyme, such as β-glucuronidase.

In certain embodiments, —Y is a peptidyl moiety.

It is understood that if —Y is a peptidyl moiety, then the release of the drug D may be triggered by an enzyme, such as protease. In certain embodiments, the protease is selected from the group consisting of cathepsin B and cathepsin K. In certain embodiments, the protease is cathepsin B. In certain embodiments, the protease is cathepsin K.

In certain embodiments, —Y is a peptidyl moiety, such as a dipeptidyl, tripeptidyl, tetrapeptidyl, pentapeptidyl or hexapeptidyl moiety. In certain embodiments, —Y is a dipeptidyl moiety. In certain embodiments, —Y is a tripeptidyl moiety. In certain embodiments, —Y is a tetrapeptidyl moiety. In certain embodiments, —Y is a pentapeptidyl moiety. In certain embodiments, —Y is a hexapeptidyl moiety.

In certain embodiments, —Y is a peptidyl moiety selected from the group consisting of:

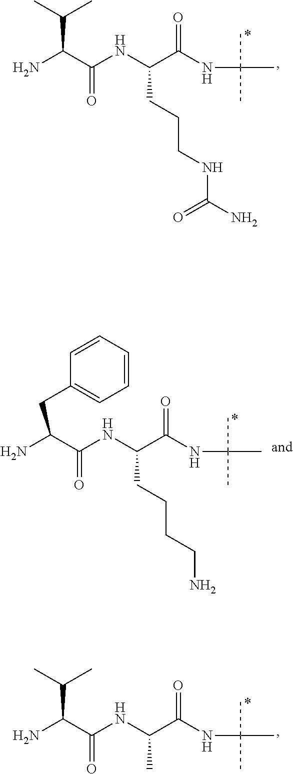

wherein the dashed line marked with an asterisk indicates the attachment to -A-.

In certain embodiments, —Y is

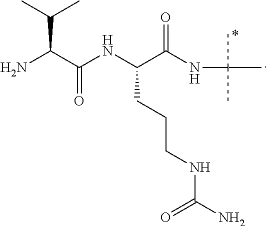

In certain embodiments, —Y is

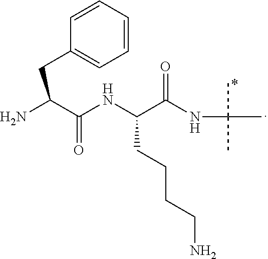

In certain embodiments, —Y is

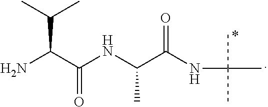

As -L¹- is connected to -D⁺ through a quaternary ammonium cation, it is understood that this linkage may not be reversible per se, but that the conjugates of the present invention may undergo a 1,(4+2p′) elimination reaction wherein p′ is at least one, which renders this linkage reversible.

In certain embodiments, -L¹- is further substituted with one or more substituents. In certain embodiments, -L¹- is not further substituted.

In certain embodiments all moieties -L²- of the conjugate of the present invention are identical. In certain embodiments the conjugate of the present invention comprises more than one type of -L²-, such as two, three, four or five different moieties -L²-. Such more than one type of -L²- may be connected to only one type of -L¹- or may be connected to more than one type of -L¹-.

In certain embodiments, -L²- is a chemical bond. In certain embodiments, -L²- is a spacer moiety.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl and C₂₋₅₀ alkynyl; wherein -T′-, C₁₋₅₀ alkyl, C₂₋₅₀ alkenyl and C₂₋₅₀ alkynyl are optionally substituted with one or more —R^{y2}, which are the same or different and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})— and —OC(O)N(R^{y3})—;

—R^{y1} and —R^{y1a} are independently selected from the group consisting of —H, -T′, C₁₋₅₀ alkyl, C₂₋₅₀ alkenyl and C₂₋₅₀ alkynyl; wherein -T′, C₁₋₅₀ alkyl, C₂₋₅₀ alkenyl and C₂₋₅₀ alkynyl are optionally substituted with one or more —R^{y2}, which are the same or different, and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y2}, which are the same or different;

each —R^{y2} is independently selected from the group consisting of halogen, —CN, oxo (═O), —C(O)OR^{y5}, —OR^{y5}, —C(O)R^{y5}, —C(O)N(R^{y5})(R^{y5a}), —S(O)₂N(R^{y5})(R^{y5a}), —S(O)N(R^{y5})(R^{y5a}), —S(O)₂R^{y5}, —S(O)R^{y5}, —N(R^{y5})S(O)₂N(R^{y5})(R^{y5a}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5})(R^{y5a}), —OC(O)N(R^{y5})(R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₂₀ alkyl, C₂₋₂₀ alkenyl, and C₂₋₂₀ alkynyl are optionally substituted with one or more —R^{y2}, which are the same or different and wherein C₁. alkyl, C₂₋₂₀ alkenyl, and C₂₋₂₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1}a are independently selected from the group consisting of —H, -T′, C₁₋₁₀ alkyl, C₂₋₁₀ alkenyl, and C₂₋₁₀ alkynyl; wherein -T′, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T′ is independently optionally substituted with one or more —R^{y2}, which are the same or different;

—R^{y2} is selected from the group consisting of halogen, —CN, oxo (═O), —C(O)OR^{y5}, —OR^{y5}, —C(O)R^{y5}, —C(O)N(R^{y5})(R^{y5a}), —S(O)₂N(R^{y5})(R^{y5a}), —S(O)N(R^{y5})(R^{y5a}), —S(O)₂R^{y5}, —S(O)R^{y5}, —N(R^{y5})S(O)₂N(R^{y5a})(R^{y5b}), —SR^{y5}, —N(R^{y5})(R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a})(R^{y}s1), —OC(O)N(R^{y5})(R^{y5a}) and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently selected from the group consisting of —H, -T′, C₁₋₁₀ alkyl, C₂₋₁₀ alkenyl and C₂₋₁₀ alkynyl;

each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl;

each —R^{y2} is independently selected from the group consisting of halogen, and C₁₋₆ alkyl; and

each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

In certain embodiments, -L²- is a C₁₋₂₀ alkyl chain, which is optionally interrupted by one or more groups independently selected from the group consisting of —O—, -T′- and —C(O)N(R^{y1})—; and which C₁₋₂₀ alkyl chain is optionally substituted with one or more groups independently selected from the group consisting of —OH, -T′ and —C(O)N(R^{y6}R^{y6a}); wherein —R^{y1}, —R^{y6}, —R^{y6a} are independently selected from the group consisting of H and C₁₋₄ alkyl and wherein T′ is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl.

In certain embodiments, -L²- has a molecular weight in the range of from 14 g/mol to 750 g/mol.

In certain embodiments, -L²- comprises a moiety selected from the group consisting of.

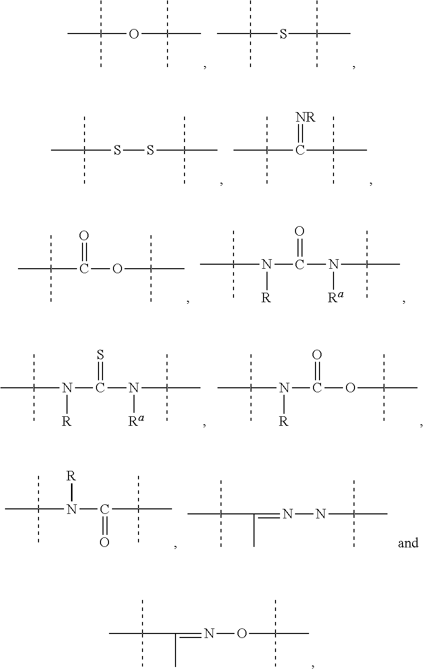

wherein dashed lines indicate attachment to -L¹-, the remainder of -L²- or Z, respectively; and —R and —R^{a} are independently selected from the group consisting of —H, methyl, ethyl, n-propyl, isopropyl, n-buty, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl.

In general, -L²- may be attached to -L¹- at any position where one hydrogen given by —R¹, —R^{1a}, —R², —R³, —R⁴, —R⁵, —R⁶, —R⁷, —R⁸, —R⁹, —R¹⁰, —R^{10a}, —R¹¹, —R¹², —R¹³, —R¹⁴, —R¹⁵, —R^{15a}, —R¹⁶, —R¹⁷, —R¹⁸, —R¹⁹, —R^{19a}, —R²⁰, —R²¹, —R^{21a}, —R²², —R²³, —R²⁴, —R^{24a}, —R^{2s}, —R²⁶, —R^{26a}, —R²⁷, —R²⁸, —R²⁹, —R^{29a} or by a peptidyl moiety, such as valine-citrulline, phenylalanine-lysine or valine-alanine is replaced by -L²-.

In certain embodiments, one hydrogen given by —R¹ is replaced by -L²-. In certain embodiments, one hydrogen given by —R³ is replaced by -L²-. In certain embodiments, one hydrogen given by —R⁴ is replaced by -L²-. In certain embodiments, one hydrogen given by —R⁵ is replaced by -L²-. In certain embodiments, one hydrogen given by —R⁶ is replaced by -L²-. In certain embodiments, one hydrogen given by —R⁷ is replaced by -L²-. In certain embodiments, one hydrogen given by —R⁸ is replaced by -L²-. In certain embodiments, one hydrogen given by —R⁹ is replaced by -L²-. In certain embodiments, one hydrogen given by —R¹⁰ is replaced by -L²-. In certain embodiments, one hydrogen given by —R^{10a} is replaced by -L²-. In certain embodiments, one hydrogen given by —R¹¹ is replaced by -L²-. In certain embodiments, one hydrogen given by —R¹² is replaced by -L²-. In certain embodiments, one hydrogen given by —R¹³ is replaced by -L²-. In certain embodiments, one hydrogen given by —R¹⁴ is replaced by -L²-. In certain embodiments, one hydrogen given by —R¹⁵ is replaced by -L²-. In certain embodiments, one hydrogen given by —R^{15a} is replaced by -L²-. In certain embodiments, one hydrogen given by —R¹⁶ is replaced by -L²-. In certain embodiments, one hydrogen given by —R¹⁷ is replaced by -L²-. In certain embodiments, one hydrogen given by —R¹⁸ is replaced by -L²-. In certain embodiments, one hydrogen given by —R¹⁹ is replaced by -L²-. In certain embodiments, one hydrogen given by —R^{19a} is replaced by -L²-. In certain embodiments, one hydrogen given by —R²⁰ is replaced by -L²-. In certain embodiments, one hydrogen given by —R²¹ is replaced by -L²-. In certain embodiments, one hydrogen given by —R^{21a} is replaced by -L²-. In certain embodiments, one hydrogen given by —R²² is replaced by -L²-. In certain embodiments, one hydrogen given by —R²³ is replaced by -L²-. In certain embodiments, one hydrogen given by —R²⁴ is replaced by -L²-. In certain embodiments, one hydrogen given by —R^{24a} is replaced by -L²-. In certain embodiments, one hydrogen given by —R²⁵ is replaced by -L²-. In certain embodiments, one hydrogen given by —R²⁶ is replaced by -L²-. In certain embodiments, one hydrogen given by —R^{26a} is replaced by -L²-. In certain embodiments, one hydrogen given by —R²⁷ is replaced by -L²-. In certain embodiments, one hydrogen given by —R²⁸ is replaced by -L²-. In certain embodiments, one hydrogen given by —R²⁹ is replaced by -L²-. In certain embodiments, one hydrogen given by —R^{29a} is replaced by -L²-.

In certain embodiments, one hydrogen given by —R^{1a} is replaced by -L²- and -L¹- is of formula (Ia):

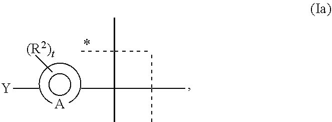

wherein
the unmarked dashed line indicates the attachment to the N⁺ of -D⁺, the dashed line marked with an asterisk indicates the attachment to -L²-; and
—R¹, -A-, —Y, each —R² and t are defined as in formula (I).

In certain embodiments, one hydrogen given by —R² is replaced by -L²- and -L¹- is of formula (Ib):

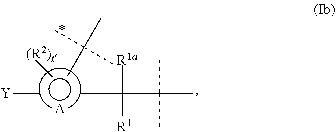

wherein
the unmarked dashed line indicates the attachment to the N⁺ of -D⁺, the dashed line marked with an asterisk indicates the attachment to -L²-;
—R¹, —R^{1a}, -A-, —Y and each —R² are defined as in formula (I); and
t′ is selected from the group consisting of 0, 1, 2, 3, 4 and 5.

In certain embodiments, t′ of formula (Ib) is 0. In certain embodiments, t′ of formula (Ib) is 1. In certain embodiments, t′ of formula (Ib) is 2. In certain embodiments, t′ of formula (Ib) is 3. In certain embodiments, t′ of formula (Ib) is 4. In certain embodiments, t′ of formula (Ib) is 5.

In certain embodiments, Z is a polymeric moiety.

In certain embodiments, Z is a C₈₋₂₄ alkyl.

In certain embodiments, Z is water-soluble.

In certain embodiments, Z is a water-soluble polymeric moiety.

If Z is a water-soluble polymeric moiety, such polymeric moiety has a molecular weight ranging from and including 1 kDa to 1000 kDa. In certain embodiments, Z has a molecular weight ranging from and including 5 kDa to 1000 kDa. In certain embodiments, Z has a molecular weight ranging from and including 5 kDa to 500 kDa. In certain embodiments, Z has a molecular weight ranging from and including 10 kDa to 250 kDa. In certain embodiments, Z has a molecular weight ranging from and including 10 kDa to 150 kDa. In certain embodiments, Z has a molecular weight ranging from and including 12 kDa to 100 kDa. In certain embodiments, Z has a molecular weight ranging from and including 15 kDa to 80 kDa. In certain embodiments, Z has a molecular weight ranging from and including 10 kDa to 80 kDa.

In certain embodiments, Z has a molecular weight of about 80 kDa. In certain embodiments, Z has a molecular weight of about 70 kDa. In certain embodiments, Z has a molecular weight of about 60 kDa. In certain embodiments, Z has a molecular weight of about 50 kDa. In certain embodiments, Z has a molecular weight of about 40 kDa. In certain embodiments, Z has a molecular weight of about 30 kDa. In certain embodiments, Z has a molecular weight of about 20 kDa. In certain embodiments, Z has a molecular weight of about 10 kDa. In certain embodiments, Z has a molecular weight of about 5 kDa.

In certain embodiments, Z is a water-soluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments, Z is a water-soluble polymeric moiety comprising a protein, such as a protein selected from the group consisting of carboxyl-terminal peptide of the chorionic gonadotropin as described in US 2012/0035101 A1 which are herewith incorporated by reference; albumin; XTEN sequences as described in WO 2011123813 A2 which are herewith incorporated by reference; proline/alanine random coil sequences as described in WO 2011/144756 A1 which are herewith incorporated by reference; proline/alanine/serine random coil sequences as described in WO 2008/155134 A1 and WO 2013/024049 A1 which are herewith incorporated by reference; and Fc-fusion proteins.

In certain embodiments, Z is a polysarcosine. In certain embodiments, Z comprises poly(N-methylglycine). In certain embodiments, Z comprises a random coil protein moiety.

In certain embodiments, such random coil protein moiety comprises at least 25 amino acid residues and at most 2000 amino acids. In certain embodiments, such random coil protein moiety comprises at least 30 amino acid residues and at most 1500 amino acid residues. In certain embodiments, such random coil protein moiety comprises at least 50 amino acid residues and at most 500 amino acid residues.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments at least 99% of the total number of amino acids forming said random coil protein moiety are selected from alanine and proline. In certain embodiments, at least 10%, but less than 75%, in certain embodiments less than 65% of the total number of amino acid residues of such random coil protein moiety are proline residues. In certain embodiments, such random coil protein moiety is as described in WO 2011/144756 A1, which is hereby incorporated by reference in its entirety. In certain embodiments, Z comprises at least one moiety selected from the group consisting of SEQ ID NO:1, SEQ ID NO:2, SEQ ID NO:3, SEQ ID NO:4, SEQ ID NO:5, SEQ ID NO:6, SEQ ID NO: 7, SEQ ID NO: 8, SEQ ID NO: 9, SEQ ID NO:10, SEQ ID NO:11, SEQ ID NO:12, SEQ ID NO:13, SEQ ID NO: 14, SEQ ID NO:15, SEQ ID NO:16, SEQ ID NO:17, SEQ ID NO:51 and SEQ ID NO:61 as disclosed in WO2011/144756 which are hereby incorporated by reference. A moiety comprising such random coil protein comprising alanine and proline will be referred to as “PA” or “PA moiety”.

Accordingly, Z comprises a PA moiety.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments at least 99% of the total number of amino acids forming said random coil protein moiety are selected from alanine, serine and proline. In certain embodiments, at least 4%, but less than 40% of the total number of amino acid residues of such random coil protein moiety are proline residues. In certain embodiments, such random coil protein moiety is as described in WO 2008/155134 A1, which is hereby incorporated by reference in its entirety. In certain embodiments, Z comprises at least one moiety selected from the group consisting of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:40, SEQ ID NO:42, SEQ ID NO:44, SEQ ID NO:46, SEQ ID NO:50, SEQ ID NO:52, SEQ ID NO:54 and SEQ ID NO:56 as disclosed in WO 2008/155134 A1, which are hereby incorporated by reference. A moiety comprising such random coil protein moiety comprising alanine, serine and proline will be referred to as “PAS” or “PAS moiety”.

Accordingly, Z comprises a PAS moiety.

In certain embodiments, Z comprises a random coil protein moiety of which at least 80%, in certain embodiments at least 85%, in certain embodiments at least 90%, in certain embodiments at least 95%, in certain embodiments at least 98% and in certain embodiments 99% of the total number of amino acids forming said random coil protein moiety are selected from alanine, glycine, serine, threonine, glutamate and proline. In certain embodiments, such random coil protein moiety is as described in WO 2010/091122 A1 which is hereby incorporated by reference. In certain embodiments, Z comprises at least one moiety selected from the group consisting of SEQ ID NO:182, SEQ ID NO:183, SEQ ID NO:184; SEQ ID NO:185, SEQ ID NO:186, SEQ ID NO:187, SEQ ID NO:188, SEQ ID NO:189, SEQ ID NO: 190, SEQ ID NO:191, SEQ ID NO:192, SEQ ID NO:193, SEQ ID NO:194, SEQ ID NO: 195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:759, SEQ ID NO:760, SEQ ID NO:761, SEQ ID NO:762, SEQ ID NO:763, SEQ ID NO:764, SEQ ID NO:765, SEQ ID NO:766, SEQ ID NO:767, SEQ ID NO:768, SEQ ID NO:769, SEQ ID NO:770, SEQ ID NO:771, SEQ ID NO:772, SEQ ID NO:773, SEQ ID NO:774, SEQ ID NO:775, SEQ ID NO:776, SEQ ID NO:777, SEQ ID NO:778, SEQ ID NO:779, SEQ ID NO:1715, SEQ ID NO:1716, SEQ ID NO:1718, SEQ ID NO:1719, SEQ ID NO:1720, SEQ ID NO:1721 and SEQ ID NO:1722 as disclosed in WO2010/091122A1, which are hereby incorporated by reference. A moiety comprising such random coil protein moiety comprising alanine, glycine, serine, threonine, glutamate and proline will be referred to as “XTEN” or “XTEN moiety” in line with its designation in WO 2010/091122 A1.

Accordingly, Z comprises an XTEN moiety.

In certain embodiments, Z is a hyaluronic acid-based polymer.

In certain embodiments, Z is a polymeric moiety as disclosed in WO 2013/024047 A1 which is herewith incorporated by reference. In certain embodiments, Z is a polymeric moiety as disclosed in WO 2013/024048 A1 which is herewith incorporated by reference.

In certain embodiments, Z is a PEG-based polymer, such as linear, branched or multi-arm PEG-based polymer. In certain embodiments, Z is a linear PEG-based polymer.

In certain embodiments, Z is a branched C₈₋₂₄ alkyl having one, two, three, four, five or six branching points. In certain embodiments, Z is a branched C₈₋₂₄ alkyl having one, two or three branching points. In certain embodiments, Z is a branched C₈₋₂₄ alkyl having one branching point. In certain embodiments, Z is a branched C₈₋₂₄ alkyl having two branching points. In certain embodiments, Z is a branched C₈₋₂₄ alkyl having three branching points.

In certain embodiments, Z is a branched polymer. In certain embodiments, Z is a branched polymer having one, two, three, four, five or six branching points. In certain embodiments, Z is a branched polymer having one, two or three branching points. In certain embodiments, Z is a branched polymer having one branching point. In certain embodiments, Z is a branched polymer having two branching points. In certain embodiments, Z is a branched polymer having three branching points.

In certain embodiments, a branching point is selected from the group consisting of —N<, —CH< and >C<. In certain embodiments, such branched moiety Z is PEG-based.

In certain embodiments, Z is a multi-arm PEG-based polymer. In certain embodiments, Z is a multi-arm PEG-based polymer having at least 2 PEG-based arms, such as 2, 3, 4, 5, 6, 7 or 8 PEG-based arms.

In certain embodiments, Z is a branched PEG-based polymer comprising at least 10% PEG, has one branching point and two PEG-based polymer arms and has a molecular weight of about 40 kDa. Accordingly, each of the two PEG-based polymer arms has a molecular weight of about 20 kDa. In certain embodiments, the branching point is —CH<.

In certain embodiments, Z is a branched PEG-based polymer comprising at least 10% PEG, has three branching points and four PEG-based polymer arms and has a molecular weight of about 40 kDa. Accordingly, each of the four PEG-based polymer arms has a molecular weight of about 10 kDa. In certain embodiments, each of the three branching points is —CH<.

In certain embodiments, Z is water-insoluble.

In certain embodiments, Z is a water-insoluble polymeric moiety.

In certain embodiments, Z is a water-insoluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

In certain embodiments, Z is a hydrogel.

In certain embodiments, Z is a PEG-based or hyaluronic acid-based hydrogel. In certain embodiments, Z is a PEG-based hydrogel. In certain embodiments, Z is a hyaluronic acid-based hydrogel.

In certain embodiments, Z is a hydrogel as described in WO 2006/003014 A2, WO 2011/012715 A1 or WO 2014/056926 A1, which are herewith incorporated by reference in their entirety.

In certain embodiments, Z is a polymer network formed through the physical aggregation of polymer chains, which physical aggregation is preferably caused by hydrogen bonds, crystallization, helix formation or complexation. In certain embodiments, such polymer network is a thermogelling polymer.

In certain embodiments, Z comprises a moiety selected from the group consisting of:

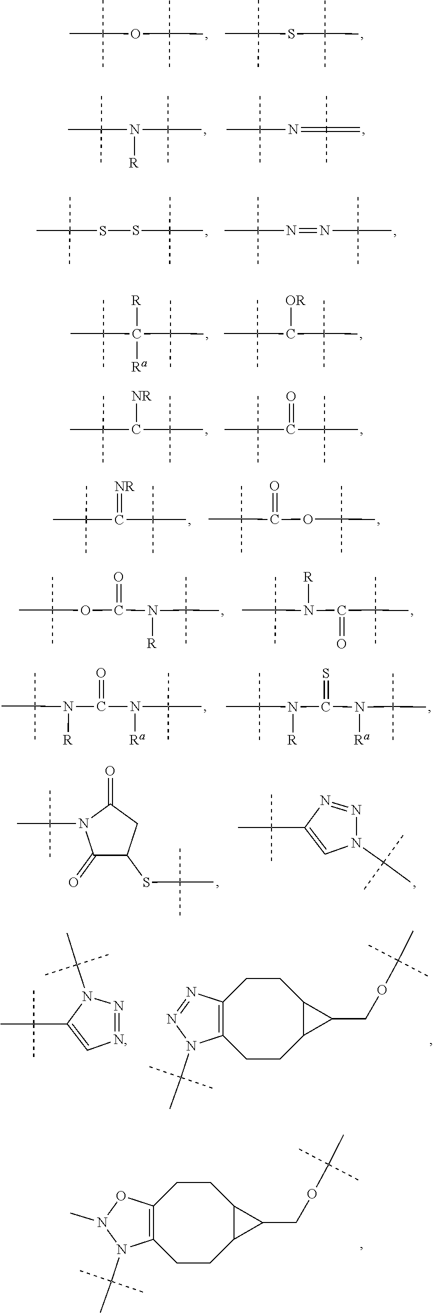

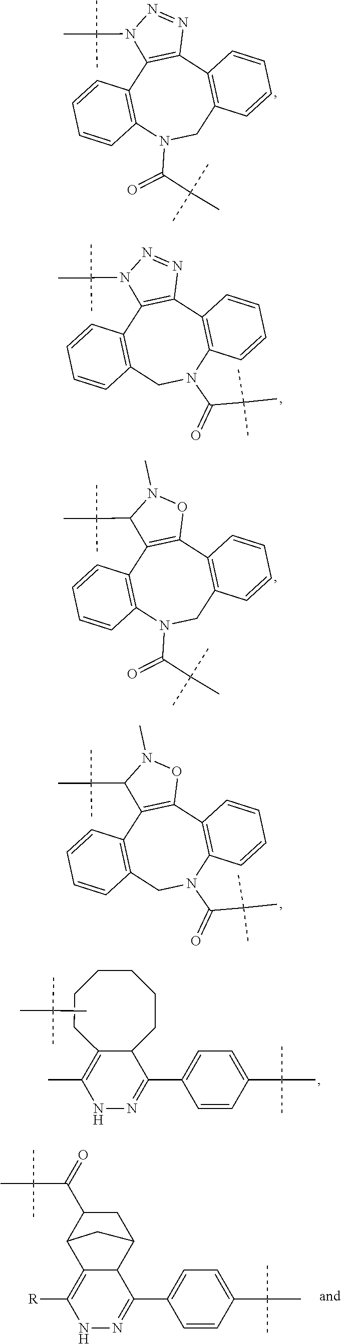

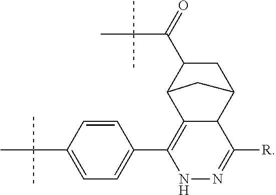

Another aspect of the present invention is a conjugate or a pharmaceutically acceptable salt thereof comprising at least one moiety -D⁺ conjugated via at least one moiety -L¹-L²- to at least one moiety Z, wherein a moiety -L¹- is conjugated to a N⁺ of a moiety -D⁺ and wherein the linkage between -D⁺ and -L¹- is reversible and wherein a moiety -L²- is conjugated to Z, wherein
each -D⁺ is independently an electron-donating heteroaromatic N⁺-comprising moiety or a quaternary ammonium cation comprising moiety of a drug D, wherein each D comprises an electron-donating heteroaromatic N or a tertiary amine;
each -L²- is independently a single bond or a spacer moiety;
each Z is independently a polymeric moiety or a C₈₋₂₄ alkyl;
each -L¹- is independently a linker moiety of formula (II):

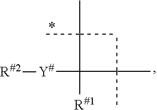

wherein
the dashed line marked with an asterisk indicates the attachment to -L²-, the unmarked dashed line indicates the attachment to the N⁺ of -D⁺;
—Y^{#}— is selected from the group consisting of —N(R^{#3})—, —O— and —S—;
—R^{#1}, —R^{#2} and —R^{#3} are independently selected from the group consisting of —H, -T^{#}, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R^{#4} which are the same or different; and wherein C₁₋₆ alkyl, C₂₋₆ alkenyl and C₂₋₆ alkynyl are optionally interrupted by one or more groups selected from the group consisting of -T^{#}-, —C(O)O—, —O—, —C(O)—, —C(O)N(R⁵)—, —S(O)₂N(R^{#5})—, —S(O)N(R⁵)—, —S(O)₂—, —S(O)—, —N(R^{#5})S(O)₂N(R^{#5a})—, —S—, —N(R^{#5})—, —OC(OR⁵)(R^{#5a})—, —N(R^{#5})C(O)N(R^{#5a})— and —OC(O)N(R^{#5})—;
   each T^{#} is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each T^{#} is independently optionally substituted with one or more —R^{#4}, which are the same or different; and
   wherein —R^{#4}, —R^{#5} and —R^{#5a} are independently selected from the group consisting of —H and C₁₋₆ alkyl; wherein C₁₋₆ alkyl is optionally substituted with one or more halogen, which are the same or different; and
each -L¹- is substituted with -L²- and optionally further substituted.

In certain embodiments, —Y^{#}— is —N(R^{#3})—. In certain embodiments, —Y^{#}— is —O—. In certain embodiments, —Y^{#}— is —S—.

In certain embodiments, —R^{#1}, —R^{#2} and —R^{#3} are independently selected from the group consisting of —H, -T^{#}, C₁₋₆ alkyl, C₂₋₆ alkenyl and C₂₋₆ alkynyl.

In certain embodiments, —R^{#1} is independently selected from the group consisting of —H, -T^{#}, C₁₋₆ alkyl, C₂₋₆ alkenyl and C₂₋₆ alkynyl. In certain embodiments, —R^{#1} is —H. In certain embodiments, —R^{#1} is -T^{#}. In certain embodiments, —R^{#1} is C₁₋₆ alkyl. In certain embodiments, —R^{#1} is C₂₋₆ alkenyl. In certain embodiments, —R^{#1} is C₂₋₆ alkynyl.

In certain embodiments, —R^{#2} is independently selected from the group consisting of —H, -T^{#}, C₁₋₆ alkyl, C₂₋₆ alkenyl and C₂₋₆ alkynyl. In certain embodiments, —R^{#2} is —H. In certain embodiments, —R² is -T^{#}. In certain embodiments, —R^{#2} is C₁₋₆ alkyl. In certain embodiments, —R^{#2} is C₂₋₆ alkenyl. In certain embodiments, —R^{#2} is C₂₋₆ alkynyl.

In certain embodiments, —R^{#3} is independently selected from the group consisting of —H, -T^{#}, C₁₋₆ alkyl, C₂₋₆ alkenyl and C₂₋₆ alkynyl. In certain embodiments, —R^{#3} is —H. In certain embodiments, —R^{#3} is -T^{#}. In certain embodiments, —R^{#3} is C₁₋₆ alkyl. In certain embodiments, —R^{#3} is C₂₋₆ alkenyl. In certain embodiments, —R^{#3} is C₂₋₆ alkynyl.

In certain embodiments, T^{#} is selected from the group consisting of phenyl, naphthyl, indenyl, indanyl, tetralinyl, C₃₋₁₀ cycloalkyl, 3- to 10-membered heterocyclyl and 8- to 11- heterobicyclyl. In certain embodiments, T^{#} is phenyl. In certain embodiments, T^{#} is naphthyl. In certain embodiments, T^{#} is indenyl. In certain embodiments, T^{#} is indanyl. In certain embodiments, T^{#} is tetralinyl. In certain embodiments, T^{#} is C₃₋₁₀ cycloalkyl. In certain embodiments, T^{#} is 3- to 10-membered heterocyclyl. In certain embodiments, T^{#} is 8- to 11-heterobicyclyl.

In certain embodiments, T^{#} is substituted with one or more —R^{#4}. In certain embodiments, T^{#} is substituted with one —R^{#4}. In certain embodiments, T^{#} is not substituted with —R^{#4}.

In certain embodiments, —R^{#4}, —R^{#5} and —R^{#5a} are independently selected from the group consisting of —H and C₁₋₆ alkyl.

In certain embodiments, —R^{#4} is selected from the group consisting of —H and C₁₋₆ alkyl. In certain embodiments, —R^{#4} is —H. In certain embodiments, —R^{#4} is C₁₋₆ alkyl.

In certain embodiments, —R^{#5} is selected from the group consisting of —H and C₁₋₆ alkyl. In certain embodiments, —R^{#5} is —H. In certain embodiments, —R^{#5} is C₁₋₆ alkyl.

In certain embodiments, —R^{#5a} is selected from the group consisting of —H and C₁₋₆ alkyl. In certain embodiments, —R^{#5a} is —H. In certain embodiments, —R^{#5a} is C₁₋₆ alkyl.

In certain embodiments, —Y^{#}— is —O— and —R^{#2} is C₁₋₆ alkyl. In certain embodiments, —Y^{#}— is —O— and —R^{#2} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments, —Y^{#}— is —O— and —R^{#2} is methyl. In certain embodiments, —Y^{#}— is —O— and —R^{#2} is ethyl.

In certain embodiments, —Y^{#}— is —O— and —R^{#2} is C₁₋₆ alkyl, wherein C₁₋₆ alkyl is interrupted by —C(O)—.

In certain embodiments, —Y^{#}— is —N(R³)— and —R^{#2} is C₁₋₆ alkyl, wherein C₁₋₆ alkyl is interrupted by —C(O)O— and —R^{#3} is as defined in formula (II).

In certain embodiments, —Y^{#}— is —N(R³)— and —R^{#2} is C₁₋₆ alkyl, wherein C₁₋₆ alkyl is interrupted by —C(O)O— and —R^{#3} is selected from the group consisting of —H, methyl, ethyl and propyl.

In certain embodiments, -L¹- of formula (II) is of formula (IIi):

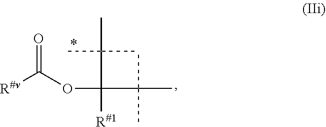

wherein
the dashed line marked with an asterisk indicates the attachment to -L²- and the unmarked dashed line indicates the attachment to the π-electron-pair-donating heteroaromatic N of -D;
—R^{#v} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R^{#1} is used as defined in formula (II).

In certain embodiments, —R^{#v} of formula (IIi) is selected from the group consisting of methyl, ethyl and propyl. In certain embodiments, —R^{#v} of formula (IIi) is methyl. In certain embodiments, —R^{#v} of formula (IIi) is ethyl. In certain embodiments, —R^{#v} of formula (IIi) is propyl.

In certain embodiments, -L¹- of formula (II) is of formula (IIii):

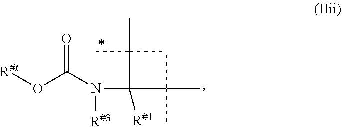

wherein
the dashed line marked with an asterisk indicates the attachment to -L²- and the unmarked dashed line indicates the attachment to the π-electron-pair-donating heteroaromatic N of -D;
—R^{#t} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R^{#1} and —R^{#3} are used as defined in formula (II).

In certain embodiments, —R^{#3} of formula (IIii) is selected from the group consisting of —H, methyl and ethyl. In certain embodiments, —R^{#3} of formula (IIii) is —H. In certain embodiments, —R^{#3} of formula (IIii) is methyl. In certain embodiments, —R^{#3} of formula (IIii) is ethyl.

In certain embodiments, —R^{#t} of formula (IIii) is selected from the group consisting of methyl, ethyl and propyl. In certain embodiments, —R^{#t} of formula (IIii) is methyl. In certain embodiments, —R^{#t} of formula (IIii) is ethyl. In certain embodiments, —R^{#t} of formula (IIii) is propyl.

In certain embodiments, -L¹- of formula (II) is of formula (IIiii):

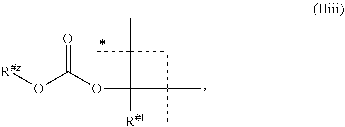

wherein
the dashed line marked with an asterisk indicates the attachment to -L²- and the unmarked dashed line indicates the attachment to the π-electron-pair-donating heteroaromatic N of -D;
—R^{#z} is selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl; and
—R^{#1} is used as defined in formula (II).

In certain embodiments, —R^{#z} of formula (IIiii) is selected from the group consisting of methyl, ethyl and propyl. In certain embodiments, —R^{#z} of formula (IIiii) is methyl. In certain embodiments, —R^{#z} of formula (IIiii) is ethyl. In certain embodiments, —R^{#z} of formula (IIiii) is propyl.

In certain embodiments, the conjugate of the present invention or the pharmaceutically acceptable salt thereof is of formula (Ia), (Ib), (Ic) or (Id):

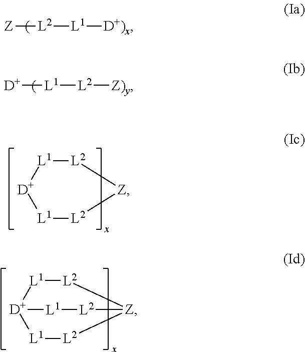

wherein
each -D⁺, -L²- and Z are defined as above and each -L¹- is independently of formula (I) or (II);
x is an integer of at least 1; and
y is an integer selected from the group consisting of 2, 3, 4 and 5.

It is understood that even though one -D⁺ can be conjugated to multiple -L¹- moieties, the drug moiety is represented by “-D⁺” and the drug by “D”.

In certain embodiments, the conjugate is of formula (Ia), (Ic) or (Id) and Z is a hydrogel. In such cases, a plurality of moieties -L²-L¹-D⁺ are conjugated to Z and it is understood that no upper limit for x can be provided.

In certain embodiments, the conjugate is of formula (Ia). In certain embodiments, the conjugate is of formula (Ib). In certain embodiments, the conjugate is of formula (Ic). In certain embodiments, the conjugate is of formula (Id). In certain embodiments, the conjugate is of formula (Ia) and Z is a hydrogel.

In certain embodiments, the conjugate is of formula (Ia), (Ic) or (Id), Z is a water-soluble polymeric moiety and x ranges from 2 to 1000, such as from 2 to 1500, such as from 2 to 1000, such as from 2 to 500, such as from 2 to 250 or such as from 2 to 100. In certain embodiments, the conjugate is of formula (Ia), (Ic) or (Id), Z is a water-soluble polymeric moiety and x is 20.

In certain embodiments, the conjugate is of formula (Ia), (Ic) or (Id), Z is a water-soluble polymeric moiety and x is 19. In certain embodiments, the conjugate is of formula (Ia), (Ic) or (Id), Z is a water-soluble polymeric moiety and x is 18. In certain embodiments, the conjugate is of formula (Ia), (Ic) or (Id), Z is a water-soluble polymeric moiety and x is 17. In certain embodiments, the conjugate is of formula (Ia), (Ic) or (Id), Z is a water-soluble polymeric moiety and x is 16. In certain embodiments, the conjugate is of formula (Ia), (Ic) or (Id), Z is a water-soluble polymeric moiety and x is 15. In certain embodiments, the conjugate is of formula (Ia), (Ic) or (Id), Z is a water-soluble polymeric moiety and x is 14. In certain embodiments, the conjugate is of formula (Ia), (Ic) or (Id), Z is a water-soluble polymeric moiety and x is 13. In certain embodiments, the conjugate is of formula (Ia), (Ic) or (Id), Z is a water-soluble polymeric moiety and x is 12. In certain embodiments, the conjugate is of formula (Ia), (Ic) or (Id), Z is a water-soluble polymeric moiety and x is 11. In certain embodiments, the conjugate is of formula (Ia), (Ic) or (Id), Z is a water-soluble polymeric moiety and x is 10. In certain embodiments, the conjugate is of formula (Ia), (Ic) or (Id), Z is a water-soluble polymeric moiety and x is 9. In certain embodiments, the conjugate is of formula (Ia), (Ic) or (Id), Z is a water-soluble polymeric moiety and x is 8. In certain embodiments, the conjugate is of formula (Ia), (Ic) or (Id), Z is a water-soluble polymeric moiety and x is 7. In certain embodiments, the conjugate is of formula (Ia), (Ic) or (Id), Z is a water-soluble polymeric moiety and x is 6. In certain embodiments, the conjugate is of formula (Ia), (Ic) or (Id), Z is a water-soluble polymeric moiety and x is 5. In certain embodiments, the conjugate is of formula (Ia), (Ic) or (Id), Z is a water-soluble polymeric moiety and x is 4. In certain embodiments, the conjugate is of formula (Ia), (Ic) or (Id), Z is a water-soluble polymeric moiety and x is 3. In certain embodiments, the conjugate is of formula (Ia), (Ic) or (Id), Z is a water-soluble polymeric moiety and x is 2.

In certain embodiments, the conjugate is of formula (Ib), Z is a water-soluble polymeric moiety and y is 1. In certain embodiments, the conjugate is of formula (Ib), Z is a water-soluble polymeric moiety and y is 2. In certain embodiments, the conjugate is of formula (Ib), Z is a water-soluble polymeric moiety and y is 3. In certain embodiments, the conjugate is of formula (Ib), Z is a water-soluble polymeric moiety and y is 4. In certain embodiments, the conjugate is of formula (Ib), Z is a water-soluble polymeric moiety and y is 5.

The conjugates of the present invention release one or more types of drug over an extended period of time, i.e. they are sustained-release conjugates. In certain embodiments, the release occurs with a release half-life ranging between 1 day and 1 month. In certain embodiments, the release occurs with a release half-life ranging between 1 day and 20 days. In certain embodiments, the release occurs with a release half-life between 1 day and 15 days. In certain embodiments the release half-life may also range from 2 to 20 days, 4 to 15 days or 3 to 6 days.

Another aspect of the present invention is a pharmaceutical composition comprising at least one conjugate of the present invention or a pharmaceutical salt thereof.

In certain embodiments, the pharmaceutical composition comprises one conjugate of the present invention or a pharmaceutical salt thereof. In certain embodiments, the pharmaceutical composition comprises two conjugates of the present invention. In certain embodiments, the pharmaceutical composition comprises three conjugates of the present invention.

Such pharmaceutical composition may have a pH ranging from pH 3 to pH 8, such as ranging from pH 4 to pH 6 or ranging from pH 4 to pH 5. In certain embodiments, the pH of the pharmaceutical composition is about 4. In certain embodiments, the pH of the pharmaceutical composition is about 4.5. In certain embodiments, the pH of the pharmaceutical composition is about 5. In certain embodiments, the pH of the pharmaceutical composition is about 5.5.

In certain embodiments, the pH of the pharmaceutical composition is 4. In certain embodiments, the pH of the pharmaceutical composition is 4.5. In certain embodiments, the pH of the pharmaceutical composition is 5. In certain embodiments, the pH of the pharmaceutical composition is 5.5.

In certain embodiments, such pharmaceutical composition is a suspension formulation.

In certain embodiments such pharmaceutical is a dry composition. It is understood that such dry composition may be obtained by drying, such as lyophilizing, a suspension composition.

If the pharmaceutical composition is a parenteral composition, suitable excipients may be categorized as, for example, buffering agents, isotonicity modifiers, preservatives, stabilizers, anti-adsorption agents, oxidation protection agents, viscosifiers/viscosity enhancing agents, anti-agglomeration agents or other auxiliary agents. However, in some cases, one excipient may have dual or triple functions. Excipient may be selected from the group consisting of

(i) Buffering agents: physiologically tolerated buffers to maintain pH in a desired range, such as sodium phosphate, bicarbonate, succinate, histidine, citrate, acetate, sulphate, nitrate, chloride, or pyruvate; antacids such as Mg(OH)₂ or ZnCO₃ may be also used;

(ii) Isotonicity modifiers: to minimize pain that can result from cell damage due to osmotic pressure differences at the injection depot; glycerin and sodium chloride are examples; effective concentrations can be determined by osmometry using an assumed osmolality of 285-315 mOsmol/kg for serum;

(iii) Preservatives and/or antimicrobials: multidose parenteral formulations require the addition of preservatives at a sufficient concentration to minimize risk of patients becoming infected upon injection and corresponding regulatory requirements have been established; typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol and benzalkonium chloride;

(iv) Stabilizers: Stabilisation is achieved by strengthening of the protein-stabilising forces, by destabilisation of the denatured state, or by direct binding of excipients to the protein; stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives; in addition, oligomers or polymers such as cyclodextrins, dextran, dendrimers, PEG or PVP or protamine or HSA may be used;

(v) Anti-adsorption agents: Mainly ionic or non-ionic surfactants or other proteins or soluble polymers are used to coat or adsorb competitively to the inner surface of the formulation's container; e.g., poloxamer (Pluronic F-68), PEG dodecyl ether (Brij 35), polysorbate 20 and 80, dextran, polyethylene glycol, PEG-polyhistidine, BSA and HSA and gelatins; chosen concentration and type of excipient depends on the effect to be avoided but typically a monolayer of surfactant is formed at the interface just above the CMC value; (vi) Oxidation protection agents: antioxidants such as ascorbic acid, ectoine, methionine, glutathione, monothioglycerol, morin, polyethylenimine (PEI), propyl gallate, and vitamin E; chelating agents such as citric acid, EDTA, hexaphosphate, and thioglycolic acid may also be used;

(vii) Viscosifiers or viscosity enhancers: retard settling of the particles in the vial and syringe and are used in order to facilitate mixing and resuspension of the particles and to make the suspension easier to inject (i.e., low force on the syringe plunger); suitable viscosifiers or viscosity enhancers are, for example, carbomer viscosifiers like Carbopol 940, Carbopol Ultrez 10, cellulose derivatives like hydroxypropylmethylcellulose (hypromellose, HPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic®), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone; such block copolymers as well as the abovementioned poloxamers may exhibit reverse thermal gelation behavior (fluid state at room temperature to facilitate administration and gel state above sol-gel transition temperature at body temperature after injection);

(viii) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue; a spreading agent such as but not limited to hyaluronidase temporarily decreases the viscosity of the extracellular matrix and promotes diffusion of injected drugs;

(ix) Anti-agglomeration agents, such as propylene glycol; and

(x) Other auxiliary agents: such as wetting agents, viscosity modifiers, antibiotics, hyaluronidase; acids and bases such as hydrochloric acid and sodium hydroxide are auxiliary agents necessary for pH adjustment during manufacture.

In another aspect, the present invention relates to a conjugate of the present invention or a pharmaceutical composition comprising a conjugate of the present invention for use as a medicament.

In another aspect, the present invention relates to a conjugate or a pharmaceutically acceptable salt thereof of the present invention or a pharmaceutical composition comprising a conjugate of the present invention for use in a method of treating a disease that can be treated with D or its pharmaceutically acceptable salt thereof.

In a further aspect, the present invention relates to a method of preventing a disease or treating a patient suffering from a disease that can be prevented or treated with D comprising administering an effective amount of the conjugate or its pharmaceutically acceptable salt thereof of the present invention or the pharmaceutical compositions comprising said conjugates to the patient.

As the present invention is applicable to all drug molecules comprising an electron-donating heteroaromatic N or a tertiary amine, it is impossible to further specify the disease that can be treated. However, it is evident to the person skilled in the art which disease can be treated with a particular conjugate.

### EXAMPLES

Materials and Methods

All materials were commercially available except where stated otherwise.

Reactions

Reactions were performed with dry solvents (CH₂Cl₂, DMF, THF) purchased from Sigma-Aldrich Chemie GmbH, Munich, Germany. Generally, reactions were stirred at room temperature and monitored by LCMS.

RP-HPLC Purification

Preparative RP-HPLC purifications were performed with a Waters 600 controller with a 2487 Dual Absorbance Detector or an Agilent Infinity 1260 preparative system using a Waters XBridge BEH300 Prep C18 10 μm, 150×30 mm column as stationary phase. Products were detected at 215 nm, 320 nm or 360 nm. Linear gradients of solvent system A (water containing 0.1% TFA v/v) and solvent system B (acetonitrile containing 0.1% TFA v/v) were used. HPLC fractions containing product were pooled and lyophilized if not stated otherwise.

Flash Chromatography

Flash chromatography purifications were performed on an Isolera One system or an Isolera Four system from Biotage AB, Sweden, using Biotage KP-Sil silica cartridges. Products were detected at 254 nm or 280 nm.

UPLC-MS Analysis

Analytical ultra-performance LC (UPLC)-MS was performed on a Waters Acquity system or an Agilent 1290 Infinity II equipped with a Waters BEH300 C18 column (2.1×50 mm, 1.7 μm particle size or 2.1×100 mm, 1.7 μm particle size); solvent A: water containing 0.04% TFA (v/v), solvent B: acetonitrile containing 0.05% TFA (v/v) coupled to a Waters Micromass ZQ or coupled to an Agilent Single Quad MS system.

### Example 1: Synthesis of Compound 1b

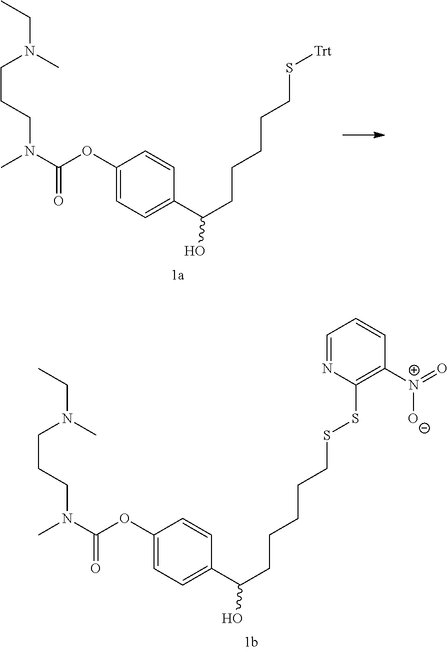

Compound 1a is synthesized as described in WO 2005/099768 A2 for compound 57b. Compound 1a (1.0 eq) and 3-nitro-2-pyridinesulfenyl chloride (1.5 eq) are stirred for 2 h under nitrogen and the volatiles removed in vacuo. This is purified to give compound 1b as the TFA salt.

### Example 2: Synthesis of Compound 2

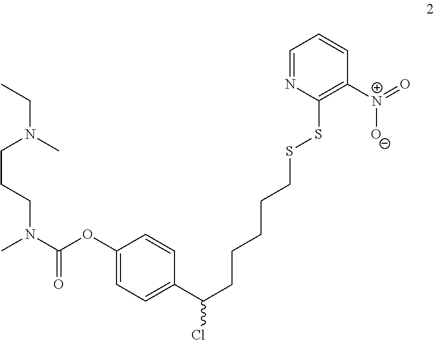 [SMILES: CCN(C)CCCN(C)C(=O)Oc1ccc(C(Cl)CCCCCSSc2ncccc2[N+](=O)[O-])cc1]

Compound 1b (1.0 eq) is dissolved in DMF and under cooling in an ice-bath, SOCl₂ (0.2 eq) in DCM is added dropwise. The reaction mixture is allowed to warm to rt and stirred for 30 minutes. The reaction mixture is quenched by addition of water before extraction into ethyl acetate. The organics are dried over MgSO₄, filtered and the solvents are removed in vacuo.

### Example 3: Synthesis of Compound 3b

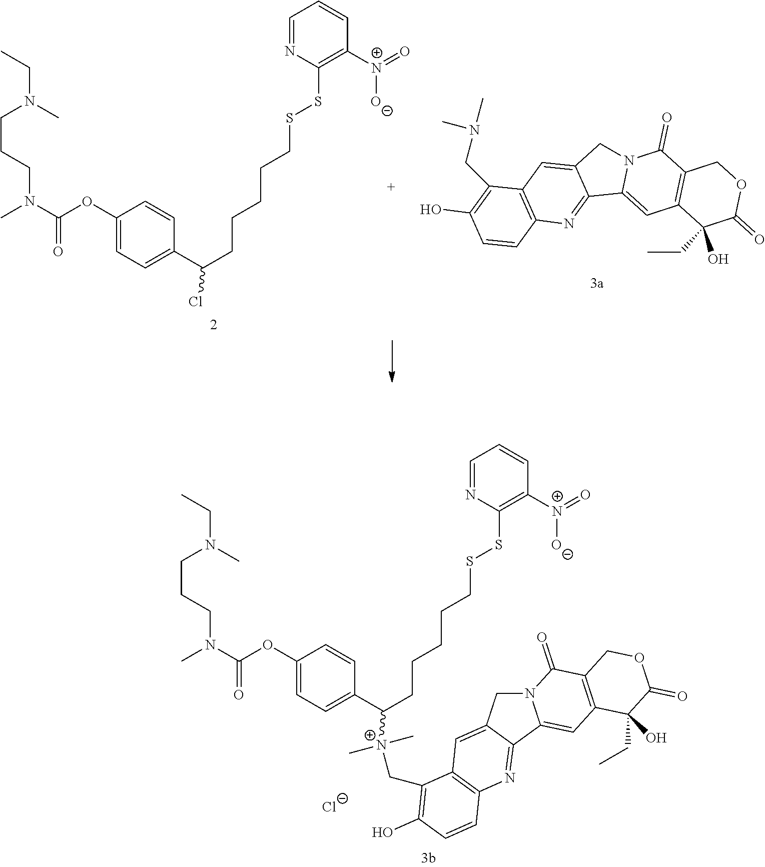 [SMILES: CCN(C)CCCN(C)C(=O)Oc1ccc(C(Cl)CCCCCSSc2ncccc2[N+](=O)[O-])cc1 ; CC[C@@]1(O)C(=O)OCc2c1cc1n(c2=O)Cc2cc3c(CN(C)C)c(O)ccc3nc2-1 ; CCN(C)CCCN(C)C(=O)Oc1ccc(C(CCCCCSSc2ncccc2[N+](=O)[O-])[N+](C)(C)Cc2c(O)ccc3nc4c(cc23)Cn2c-4cc3c(c2=O)COC(=O)[C@]3(O)CC)cc1 ; [Cl-]]

Compound 2 (1.0 eq) is combined with topotecan (3a) (1.0 eq) in DMF. Under stirring at rt, TBAI (0.2 eq) is added before addition of DIPEA (2.0 eq) before stirring at rt for 18 h. The solvent is then removed in vacuo and the product is purified by prep-HPLC.

### Example 4: Synthesis of Compound 4

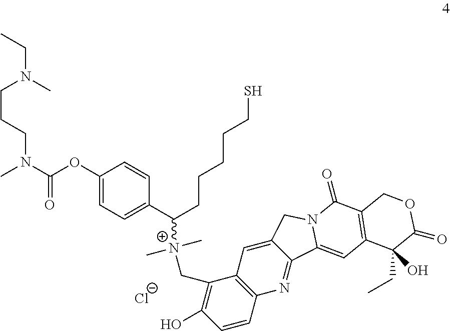 [SMILES: CCN(C)CCCN(C)C(=O)Oc1ccc(C(CCCCCS)[N+](C)(C)Cc2c(O)ccc3nc4c(cc23)Cn2c-4cc3c(c2=O)COC(=O)[C@]3(O)CC)cc1 ; [Cl-]]

Compound 3b is stirred in 10 mM DTT and 25 mM acetate buffer at pH 4.2 for 30 minutes at 4° C. before purification by prep-HPLC.

### Example 5: Synthesis of Compound 5b

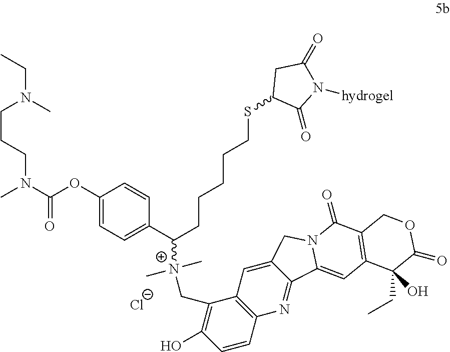

The maleimide functionalized hydrogel beads 5a are synthesized as described in WO 2014/056923 A1, Example 5. Hydrogel beads 5a and 4 are combined in 10 mM sodium phosphate, 5 mM Na₂EDTA, 0.01% Tween 20, pH 6.5 buffer to give compound 5b.

### Example 6: Synthesis of Compound 6b

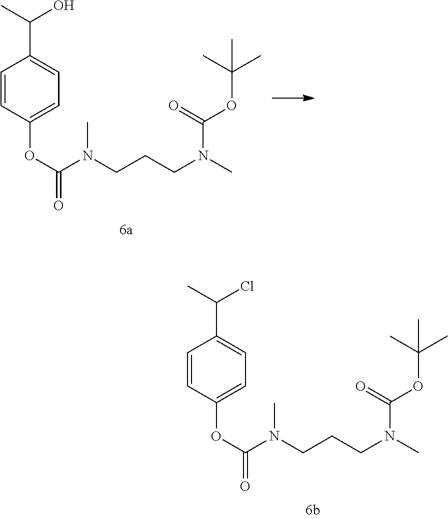 [SMILES: CC(O)c1ccc(OC(=O)N(C)CCCN(C)C(=O)OC(C)(C)C)cc1 ; CC(Cl)c1ccc(OC(=O)N(C)CCCN(C)C(=O)OC(C)(C)C)cc1]

4-(1-Hydroxyethyl)phenol (100 mg) was dissolved in 1.2 ml THF and DIPEA (0.38 mL) was added with stirring. 4-nitrophenyl chloroformate (160 mg) was dissolved in 0.3 ml of THF and added drop-wise to the reaction. The reaction was stirred for 1 h. tert-Butyl N-methyl-N-[3-(methylamino)propyl]carbamate (190 mg) was dissolved in 0.3 ml of THF and added drop-wise to the reaction. The reaction was stirred for 40 min. The reaction was quenched with 0.38 ml TFA and diluted with water (2 ml) and 6a was purified by RP-HPLC.

Yield: 193 mg (73%)

MS: m/z 367.12=[M+H]⁺, (calculated=367.22).

Chlorotriazine (79 mg, 0.43 mmol) and DMF (87 μL) were stirred in a 10 ml flask for 15 min. DCM (1 mL) was added. Sodium bicarbonate (131 mg, 1.56 mmol) was added. 6a (150 mg, 0.41 mmol) was dissolved in 0.3 ml dichloromethane and added drop-wise. After 2.5 h the reaction was diluted with 10 ml DCM and 8 ml water. The organic phase was washed with saturated bicarbonate, 1 M HCl and brine (1×8 ml, each). The organic phase was dried (MgSO₄) and concentrated in vacuo. The product was purified by flash chromatography using heptane/ethyl acetate as eluent.

Yield: 77 mg (49%)

### Example 7: Synthesis of Compound 7a and 7b

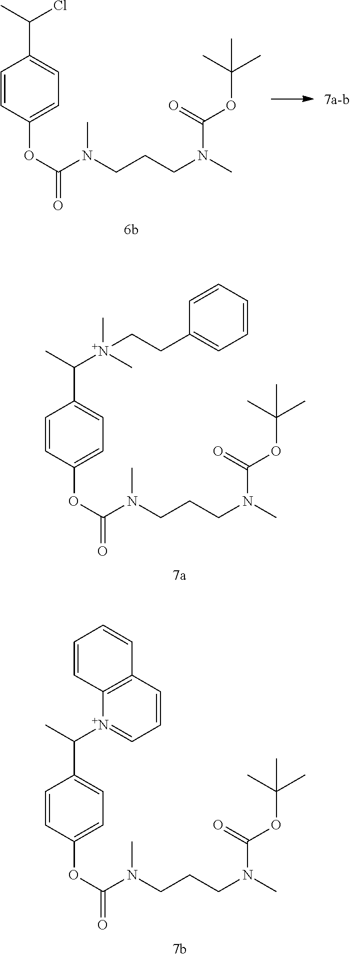 [SMILES: CC(Cl)c1ccc(OC(=O)N(C)CCCN(C)C(=O)OC(C)(C)C)cc1 ; CC(c1ccc(OC(=O)N(C)CCCN(C)C(=O)OC(C)(C)C)cc1)[N+](C)(C)CCc1ccccc1 ; CC(c1ccc(OC(=O)N(C)CCCN(C)C(=O)OC(C)(C)C)cc1)[n+]1cccc2ccccc21]

6b (17 mg, 0.04 mmol) and either N,N-dimethyl-N-phenethylamine (6 mg; 0.04 mmol) for 7a, or quinoline (26 mg; 0.2 mmol) for 7b, were dissolved in DMF (0.2 mL), and TBAI (7.4 mg, 0.02 mmol) and DIPEA (17.5 μL, 0.10 mmol) were added. The reaction was stirred for 6.5 days (7a) or 4 days (7b) and the product purified by RP-HPLC.

Yield (7a): 1.5 mg (7%); MS: m/z 498.24=[M]⁺, (calculated=498.33).

Yield (7b): 1 mg (5%); MS: m/z 478.23=[M], (calculated=478.27).

### Example 8: Synthesis of Compound 8a and 8b

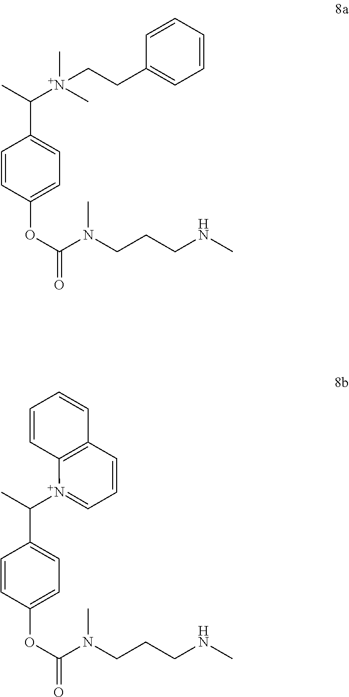 [SMILES: CNCCCN(C)C(=O)Oc1ccc(C(C)[N+](C)(C)CCc2ccccc2)cc1 ; CNCCCN(C)C(=O)Oc1ccc(C(C)[n+]2cccc3ccccc32)cc1]

7a (1.5 mg) or 7b (1 mg) was dissolved in DCM (0.5 mL), and TFA (0.5 mL) was added with stirring. After 1 h the volatiles were removed in vacuo and the residue dried yielding the TFA salt of 8a or 8b, respectively in quantitative yield.

### Example 9: In Vitro Release Kinetics

The cleavage rate of the reversible bond from conjugates 8a-b was monitored at pH 7.4 and 37° C. in aqueous buffer (pH 7.4, 48 mM sodium phosphate, 20% acetonitrile). Disappearance of the conjugate was determined by LCMS (UV detection) and fitted with curve fitting software to obtain the half-life of the release.

| Compound | t_{1/2} (pH 7.4) | Released product |
|---|---|---|
| 8a | 1.3 h | N,N-dimethyl-N-phenethylamine |
| 8b | 1.8 h | quinoline |

### Example 10: Synthesis of Compound 10

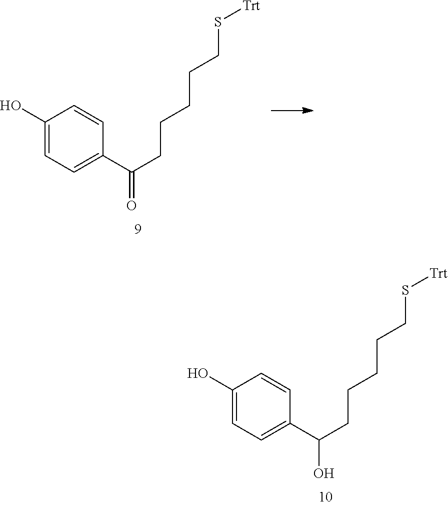

Compound 9 is synthesized as described in WO 2005/099768 A2 for compound 55b. Compound 9 (1.0 eq) is dissolved in THF and lithium borohydride (3.0 eq.) is added in an atmosphere of nitrogen. The reaction is stirred for 24 h. The product is purified by flash chromatography to give 10.

### Example 11: Synthesis of Compounds 11e and 11f

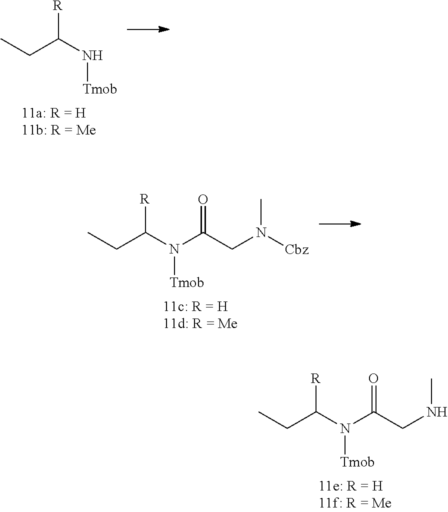

To a solution of n-propylamine (1 eq) and NaCNBH₃ (1.1 eq) in methanol is added 2,4,6-trimethoxybenzaldehyde (0.92 eq) portion wise. The mixture is stirred at rt for 90 min, acidified with 3 M HCl and stirred for a further 15 min. The reaction mixture is added to saturated bicarbonate solution and extracted 5× with DCM. The combined organic phases are dried over sodium sulfate and the solvents are evaporated in vacuo and the product further dried yielding 11a.

The synthesis of 11b is performed accordingly to 11a using sec-butylamine instead of n-propylamine.

11a (1 eq.) and Cbz-sarcosine (1.1 eq) are dissolved in DMF and PyBOP (1.1 eq) and DIPEA (3 eq) are added. After 2 h the reaction is diluted with ethyl acetate. The organic phase is washed with sodium bicarbonate, 1 M HCl, and brine, and the product purified by flash chromatography to give 11c.

The synthesis of 11d is performed accordingly to 11c using 11b instead of 11a. 11c (1 eq) is dissolved in THF. 10% Palladium on activated charcoal (0.1 eq w/w) is added and the reaction stirred in an atmosphere of hydrogen. After 3 h the reaction is filtered through celite, and the filtrate concentrated in vacuo to give 11e.

The synthesis of 11f is performed accordingly to 11e using 11d instead of 11c.

### Example 12: Synthesis of Compounds 12a-c

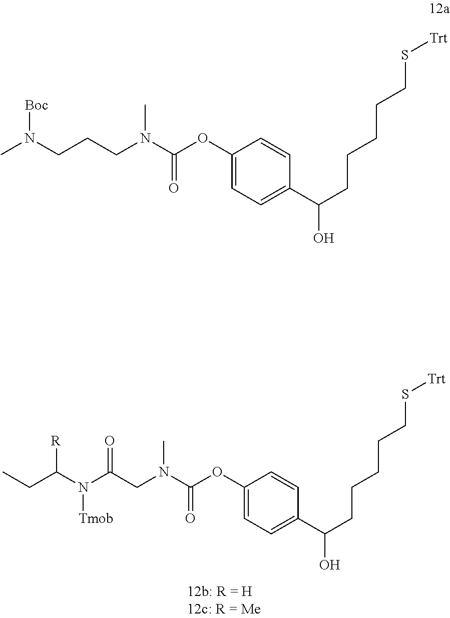

(1 eq) is dissolved in THF and DIPEA (5 eq) is added with stirring. 4-nitrophenyl chloroformate (1.1 eq) is dissolved in THF and added drop-wise to the reaction while stirring. After 1 h 11e (1.5 eq) is added to the reaction while stirring. After 1 h the reaction is quenched with acetic acid, and the product is purified by flash chromatography to give 12b.

The synthesis of 12c is performed accordingly to 12b using 11f instead of 11e.

The synthesis of 12a is performed accordingly to 12b using tert-butyl N-methyl-N-[3-(methylamino)propyl]carbamate instead of 11e.

### Example 13: Synthesis of Compounds 13a-c

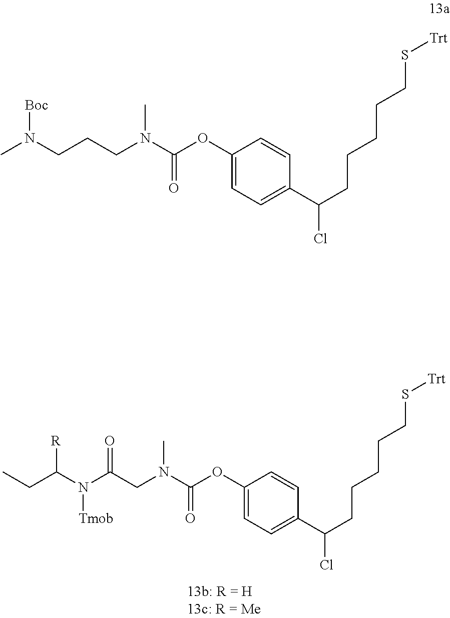

Chlorotriazine (1.05 eq) and DMF (2.7 eq) are stirred for 15 min. DCM is added. Sodium bicarbonate (3.8 eq) is added. 12a (1 eq) is dissolved in DCM and added drop-wise. After 3 h the reaction is diluted with DCM and water. The organic phase is washed with saturated bicarbonate, 1 M HCl and brine. The organic phase is dried (MgSO₄) and concentrated in vacuo. The product is purified by flash chromatography giving 13a.

The synthesis of 13b is performed accordingly to 13a using 12b instead of 12a.

The synthesis of 13c is performed accordingly to 13a using 12c instead of 12a.

### Example 14: Synthesis of Compounds 14a-c

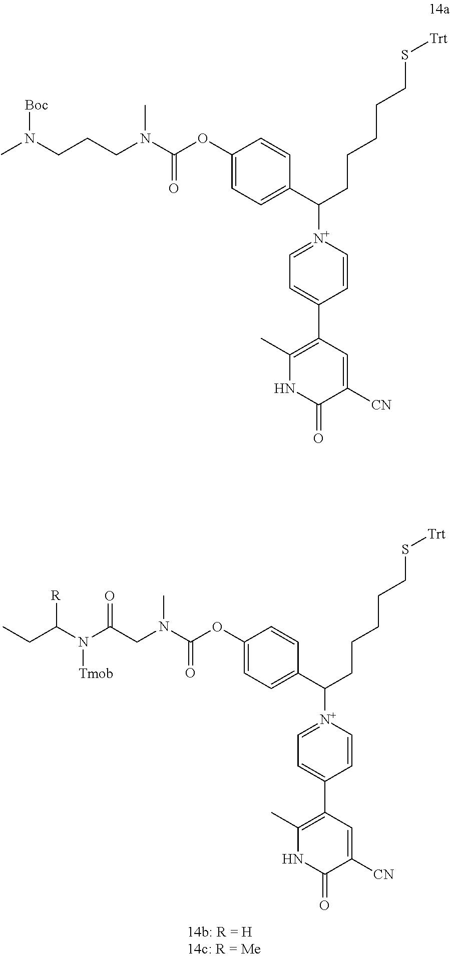

Compound 13a (1.2 eq) is combined with milrinone (1.0 eq) in DMF. Under stirring at rt, TBAI (0.2 eq) is added before addition of DIPEA (2.0 eq) before stirring at rt for 24 h. The solvent is then removed in vacuo and the product is purified by RP-HPLC giving 14a.

The synthesis of 14b is performed accordingly to 14a using 13b instead of 13a.

The synthesis of 14c is performed accordingly to 14a using 13c instead of 13a.

### Example 15: Synthesis of Compounds 15a-c

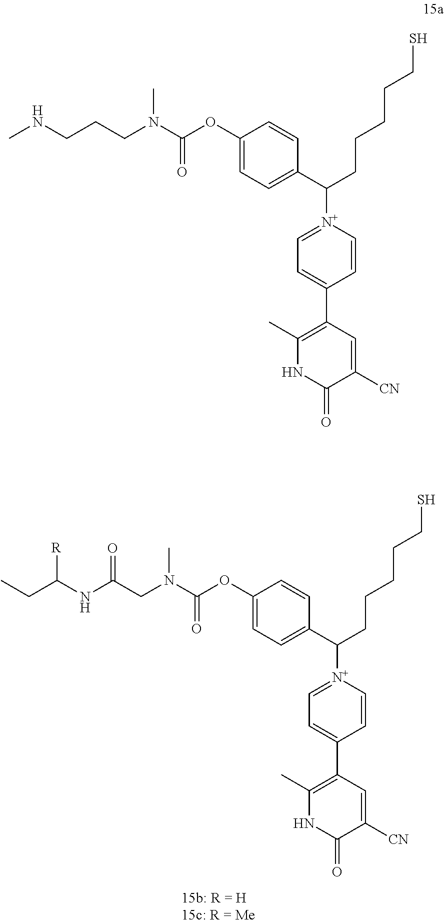

14a is dissolved in hexafluoroisopropanol/water/triethylsilane (39:1:1) and TFA is added (10% v/v). The reaction is stirred at rt for 3h. The solvent is then removed in vacuo and the product is purified by RP-HPLC giving 15a as TFA salt.

The synthesis of 15b is performed accordingly to 15a using 14b instead of 14a.

The synthesis of 15c is performed accordingly to 15a using 14c instead of 14a.

### Example 16: Synthesis of Compounds 16a-c

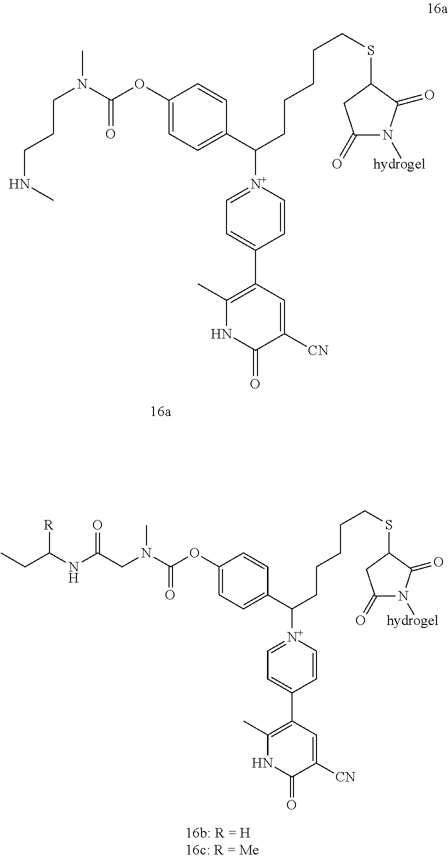

Hydrogel beads 5a and compound 15a are combined in 10 mM sodium phosphate, 5 mM Na₂EDTA, 0.01% Tween 20, pH 6.5 buffer to give compound 16a.

The synthesis of 16b is performed accordingly to 16a using 15b instead of 15a.

The synthesis of 16c is performed accordingly to 16a using 15c instead of 15a.

### Example 17: Synthesis of Compound 17

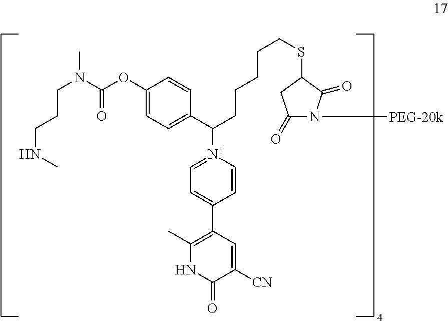

A solution of compound 16a (4.5 eq) in acetonitrile/water is mixed with 4-arm PEG 20 kDa maleimide (1 eq) and the pH is adjusted to 7.0 by addition of pH 7.4 buffer (50 mM phosphate). The mixture is stirred at rt for 2 h and then purified by RP-HPLC to give compound 17.

### Example 18: Release of Milrinone In Vitro

Release of milrinone from compounds 16a-c and 17 is effected by incubation in 60 mM sodium phosphate buffer at pH 7.4 and 37° C. Unmodified milrinone is released as assessed by LCMS.

### Abbreviations

Boc—tert.-butyloxycarbonyl
Cbz—benzyloxycarbonyl
DCM—dichloromethane
DIPEA—diisopropylethylamine
DMF—dimethylformamide
DTT—dithiothreitol
EDTA—ethylenediaminetetraacetic acid
eq—equivalent
HPLC—high performance liquid chromatography
LC-MS—liquid chromatography-coupled mass spectrometry
Me—methyl
prep-HPLC—preparative high-performance liquid chromatography
PyBOP—benzotriazol-1-yl-oxytripyrrolidinophosphonium hexafluorophosphate
RP—reversed phase
rt—room temperature
TBAI—tetrabutylammonium iodide
TFA—trifluoroacetic acid
Tmob—2,4,6-trimethoxybenzyl
Trt—trityl
UPLC—ultra performance liquid chromatography

1. A conjugate or a pharmaceutically acceptable salt thereof comprising at least one moiety -D⁺ conjugated via at least one moiety -L¹-L²- to at least one moiety Z, wherein a moiety -L¹- is conjugated to a N* of a moiety -D⁺ and wherein the linkage between -D⁺ and -L¹- is reversible and wherein a moiety -L²- is conjugated to Z, wherein
each -D⁺ is independently an electron-donating heteroaromatic N⁺-comprising moiety or a quaternary ammonium cation comprising moiety of a drug D, wherein each D comprises an electron-donating heteroaromatic N or a tertiary amine;
each -L²- is independently a single bond or a spacer moiety;
each Z is independently a polymeric moiety or a C₈₋₂₄ alkyl;
each -L¹- is independently a linker moiety of formula (I): 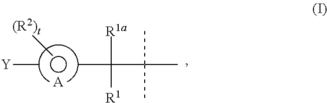
wherein
the dashed line indicates the attachment to the N⁺ of -D⁺;
t is selected from the group consisting of 0, 1, 2, 3, 4, 5 and 6;
A- is a ring selected from the group consisting of monocyclic or bicyclic aryl and heteroaryl, provided that -A- is connected to —Y and —C(R¹)(R^{1a})— via carbon atoms;
wherein said monocyclic or bicyclic aryl and heteroaryl are optionally substituted with one or more —R², which are the same or different;
—R¹, —R^{1a} and each —R² are independently selected from the group consisting of —H, —C(O)OH, -halogen, —NO₂, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})— and —OC(O)N(R⁴)—;
each -T- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein
each -T- is independently optionally substituted with one or more —R³, which are the same or different;
wherein —R³ is selected from the group consisting of —H, —NO₂, —OCH₃, —CN, —N(R⁴)(R^{4a}), —OH, —C(O)OH and C₁₋₆ alkyl; wherein C₁₋₆ alkyl is optionally substituted with one or more halogen, which are the same or different;
wherein —R⁴ and —R^{4a} are independently selected from the group consisting of —H and C₁₋₆ alkyl; wherein C₁₋₆ alkyl is optionally substituted with one or more halogen, which are the same or different;
—Y is selected from the group consisting of: 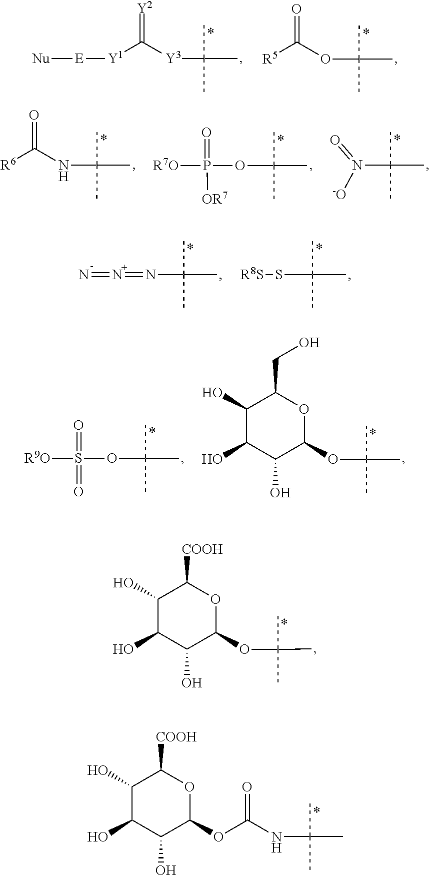
and a peptidyl moiety;
wherein the dashed line marked with an asterisk indicates the attachment to -A-;
—Nu is a nucleophile;
—Y¹— is selected from the group consisting of —O—, —C(R¹⁰)(R^{10a})—, —N(R¹¹)— and —S—;
═Y² is selected from the group consisting of ═O, ═S and ═N(R¹²);
—Y³— is selected from the group consisting of —O—, —S— and —N(R¹³)—;
-E- is selected from the group consisting of C₁₋₆ alkyl, C₂₋₆ alkenyl, C₂₋₆ alkynyl and -Q-; wherein C₁₋₆ alkyl, C₂₋₆ alkenyl, C₂₋₆ alkynyl are optionally substituted with one or more —R¹⁴, which are the same or different;
—R⁵, —R⁶, each —R⁷, —R⁸, —R⁹, —R¹⁰, —R^{10a}, —R¹¹, —R¹² and —R¹³ are independently selected from the group consisting of C₁₋₂₀ alkyl, C₂₋₂₀ alkenyl, C₂₋₂₀ alkynyl and -Q; wherein C₁₋₂₀ alkyl, C₂₋₂₀ alkenyl and C₂₋₂₀ alkynyl are optionally substituted with one or more —R¹⁴, which are the same or different; and wherein C₁₋₂₀ alkyl, C₂₋₂₀ alkenyl and C₂₋₂₀ alkynyl are optionally interrupted by one or more groups selected from the group consisting of Q, —C(O)O—, —O—, —C(O)—, —C(O)N(R¹⁵)—, —S(O)₂N(R¹⁵)—, —S(O)N(R¹⁵)—, —S(O)₂—, —S(O)—, —N(R¹⁵)S(O)₂N(R^{15a})—, —S—, —N(R¹)—, —OC(OR¹⁵)R^{15a}—, —N(R¹⁵)C(O)N(R^{15a})— and —OC(O)N(R¹⁵)—;
each Q is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein
each Q is independently optionally substituted with one or more —R¹⁴, which are the same or different;
wherein —R¹⁴, —R¹ and —R^{15a} are independently selected from the group consisting of —H and C₁₋₆ alkyl; wherein C₁₋₆ alkyl is optionally substituted with one or more halogen, which are the same or different; and
each -L¹- is substituted with -L²- and optionally further substituted.

2. The conjugate or pharmaceutically acceptable salt thereof of claim 1, wherein -D⁺ is selected from the group consisting of small molecule, medium molecule, oligonucleotide, peptide and protein drug moieties.

3. The conjugate or pharmaceutically acceptable salt thereof of claim 1 or 2, wherein Z is a polymeric moiety.

4. The conjugate or pharmaceutically acceptable salt thereof of any one of claims 1 to 3, wherein Z is a water-insoluble polymeric moiety.

5. The conjugate or pharmaceutically acceptable salt thereof of any one of claims 1 to 4, wherein Z is a water-insoluble polymeric moiety comprising a polymer selected from the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, mannans, pectins, rhamnogalacturonans, starches, hydroxyalkyl starches, hydroxyethyl starches and other carbohydrate-based polymers, xylans, and copolymers thereof.

6. The conjugate or pharmaceutically acceptable salt thereof of any one of claims 1 to 5, wherein Z is a hydrogel.

7. The conjugate or pharmaceutically acceptable salt thereof of any one of claims 1 to 6, wherein Z is a PEG-based or hyaluronic-acid based hydrogel.

8. The conjugate or pharmaceutically acceptable salt thereof of any one of claims 1 to 7, wherein Z is a PEG-based hydrogel.

9. The conjugate or pharmaceutically acceptable salt thereof of any one of claims 1 to 7, wherein Z is a hyaluronic-acid based hydrogel.

10. The conjugate or pharmaceutically acceptable salt thereof of any one of claims 1 to 3, wherein Z is a water-soluble polymeric moiety.

11. The conjugate or pharmaceutically acceptable salt thereof of any one of claims 1 to 10, wherein —Y is selected from the group consisting of 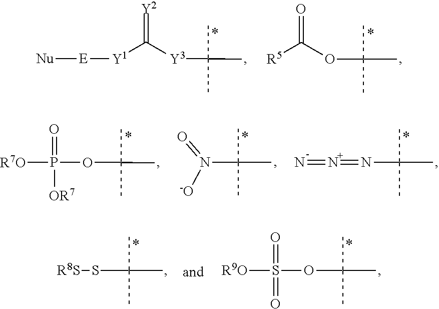
wherein —Nu, -E-, —Y¹—, ═Y², —Y³—, —R⁵, —R⁷, —R⁸ and —R⁹ are as defined in claim 1.

12. The conjugate or pharmaceutically acceptable salt thereof of any one of claims 1 to 11, wherein —Y is 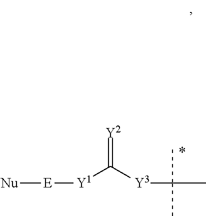
and —Nu, -E-, —Y¹—, ═Y² and —Y³— are as defined in claim 1.

13. The conjugate or pharmaceutically acceptable salt thereof of any one of claims 1 to 12, wherein —Nu is a nucleophile selected from the group consisting of primary, secondary or tertiary amine and amide.

14. The conjugate or pharmaceutically acceptable salt thereof of claim 13, wherein —Nu is a secondary amine.

15. The conjugate or pharmaceutically acceptable salt thereof of claim 13, wherein —Nu is a tertiary amine.

16. The conjugate or pharmaceutically acceptable salt thereof of any one of claims 1 to 15, wherein -E- is C₁₋₆ alkyl.

17. The conjugate or pharmaceutically acceptable salt thereof of any one of claims 1 to 16, wherein —Y¹— is —N(R¹¹)— and —R¹¹ is as defined in claim 1.

18. The conjugate or pharmaceutically acceptable salt thereof of any one of claims 1 to 17, wherein ═Y² is ═O.

19. The conjugate or pharmaceutically acceptable salt thereof of any one of claims 1 to 18, wherein —Y³— is —O—.

20. The conjugate or pharmaceutically acceptable salt thereof of any one of claims 1 to 19, wherein -L²- is a spacer moiety.

21. The conjugate or pharmaceutically acceptable salt thereof of any one of claims 1 to 20, wherein -L²- has a molecular weight in the range of from 14 g/mol to 750 g/mol.

22. The conjugate or pharmaceutically acceptable salt thereof of any one of claims 1 to 21, wherein the linkage between Z and -L²- is a stable linkage.

23. The conjugate or pharmaceutically acceptable salt thereof of any one of claims 1 to 22, wherein -L²- is a spacer moiety selected from the group consisting of -T′-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T′-, C₁₋₅₀ alkyl, C₂₋₅₀ alkenyl and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T′-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR³)(R^{y3a})—, —N(R^{y3})C(O)N(R^{y3a})—, and —OC(O)N(R^{y3})—;
wherein —R^{y1} and —R^{y1a} are independently selected from the group consisting of —H, -T′, C₁₋₅₀ alkyl, C₂₋₅₀ alkenyl and C₂₋₅₀ alkynyl; wherein -T′, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl and C₂₋₅₀ alkynyl are optionally interrupted by one or more groups selected from the group consisting of -T′-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;
each T′ is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl and 8- to 30-membered heteropolycyclyl; wherein each T′ is independently optionally substituted with one or more —R^{y2}, which are the same or different;
each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and
each —R^{y3}, —R^{y3a}, —R^{y4}, —R^{y4a}, —R^{y5}, —R^{y5a} and —R^{y5b} is independently selected from the group consisting of —H and C₁₋₆ alkyl; wherein C₁₋₆ alkyl is optionally substituted with one or more halogen, which are the same or different.

24. The conjugate or pharmaceutically acceptable salt thereof of any one of claims 1 to 23, wherein one hydrogen given by —R^{1a} is replaced by -L²- and -L¹- is of formula (Ia): 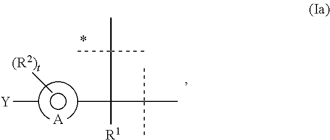
wherein
the unmarked dashed line indicates the attachment to the N⁺ of -D⁺, the dashed line marked with an asterisk indicates the attachment to -L²-; and
—R¹, -A-, —Y, each —R² and t are defined as in formula (I) of claim 1.

25. The conjugate or pharmaceutically acceptable salt thereof of any one of claims 1 to 23, wherein one hydrogen given by —R² is replaced by -L²- and -L¹- is of formula (Ib): 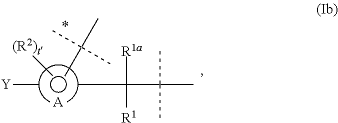
wherein
the unmarked dashed line indicates the attachment to the N⁺ of -D⁺, the dashed line marked with an asterisk indicates the attachment to -L²-;
—R¹, —R^{1a}, -A-, —Y and each —R² are defined as in formula (I) of claim 1; and
t′ is selected from the group consisting of 0, 1, 2, 3, 4 and 5.

26. A pharmaceutical composition comprising the conjugate or pharmaceutically acceptable salt thereof of any one of claims 1 to 25.

27. The conjugate or pharmaceutically acceptable salt thereof of any one of claims 1 to 25 or the pharmaceutical composition of claim 26 for use as a medicament.

28. The conjugate or pharmaceutically acceptable salt thereof of any one of claims 1 to 25 or the pharmaceutical composition of claim 26 for use in a method of treating a disease that can be treated with D.

29. A method of preventing a disease or treating a patient suffering from a disease that can be prevented or treated with D, comprising administering an effective amount of the conjugate or the pharmaceutically acceptable salt thereof of any one of claims 1 to 25 or the pharmaceutical composition of claim 26 to the patient.
